(19) United States
(12) Patent Application Publication
(10) Pub. No.: US 2010/0316639 A1
(43) Pub. Date: Dec. 16, 2010

(54) BIOMARKERS FOR IGF-1R INHIBITOR THERAPY

(75) Inventor: MARK R. LACKNER, Brisbane, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/815,548

(22) Filed: Jun. 15, 2010

Related U.S. Application Data
(60) Provisional application No. 61/187,504, filed on Jun. 16, 2009.

(51) Int. Cl. A61K 39/395; A61P 35/00; G06Q 90/00

(52) U.S. Cl. 424/133.1; 424/158.1; 424/178.1; 705/500

(57) ABSTRACT

The invention concerns the identification and validation of certain biomarkers for selecting patients for therapy with an IGF-1R inhibitor, particularly for breast and colorectal cancer.

This non-provisional application filed under 37 CFR §1.53(b), claims the benefit under 35 USC §119(e) of U.S. Provisional Application Ser. No. 61/187,504 filed on Jun. 16, 2009, which is incorporated by reference in entirety.

### FIELD OF THE INVENTION

The present invention concerns biomarkers that predict response to therapy with an insulin-like growth factor-I receptor (IGF-1R) inhibitor, particularly where the patient to be treated has breast cancer or colorectal cancer.

### BACKGROUND OF THE INVENTION

In several types of cancer, growth factors specifically bind to their receptors and then transmit growth, transformation, and/or survival signals to the tumoral cell. Over-expression of growth factor receptors at the tumoral cell surface is described, e.g., in Salomon et al., Crit. Rev. Oncol. Hematol., 19: 183 (1995); Burrow et al., J. Surg. Oncol., 69: 21 (1998); Hakam et al., Hum. Pathol., 30: 1128 (1999); Railo et al., Eur. J. Cancer, 30: 307 (1994); and Happerfield et al., J. Pathol., 183: 412 (1997). Targeting of such growth factor receptors (e.g., epidermal growth factor (EGF) receptor or HER2/neu) with humanized 4D5 (HERCEPTIN®; trastuzumab) or chimeric (C225) antibodies significantly inhibits tumoral growth in patients and increases efficacy of classical chemotherapy treatments (Carter, Nature Rev. Cancer, 1: 118 (2001); Hortobagyi, Semin. Oncol., 28: 43 (2001); Herbst et al., Semin. Oncol., 29: 27 (2002)).

Insulin-like growth factor-I (IGF-I; also called somatomedin-C) (Klapper et al., Endocrinol., 112: 2215 (1983); Rinderknecht et al., FEBS. Lett., 89: 283 (1978); U.S. Pat. No. 6,331,609; and U.S. Pat. No. 6,331,414) is a member of a family of related polypeptide hormones that also includes insulin, insulin-like growth factor-II (IGF-II) and more distantly nerve growth factor. Each of these growth factors has a cognate receptor to which it binds with high affinity, but some may also bind (albeit with lower affinity) to the other receptors as well (Rechler and Nissley, Ann. Rev. Physiol., 47: 425-42 (1985)). In the extracellular space, the IGF ligands potentially interact with four receptors and six binding proteins (Clemmons, Mol. Cell. Endocrinol., 140: 19-24 (1998)).

The IGFs exert mitogenic activity on various cell types, including tumor cells (Macaulay, Br. J. Cancer, 65:311 (1992); Ibrahim et al., Clin. Cancer Res., 11: 944s-50s (2005)), by binding to a common receptor named the insulin-like growth factor receptor-1 (IGF-1R) (Sepp-Lorenzino, Breast Cancer Research and Treatment, 47: 235 (1998)). IGF-1R (also known as EC 2.7.112, CD 221 antigen) belongs to the family of transmembrane protein tyrosine kinases (Ullrich et al., Cell, 61: 203-212, (1990), LeRoith et al., Endocrin. Rev., 16: 143-163 (1995); Traxler, Exp. Opin. Ther. Patents, 7: 571-588 (1997); Adams et al., Cell. Mol. Life. Sci., 57: 1050-1063 (2000)), and is involved in childhood growth ((Liu et al., Cell, 75: 59-72 (1993); Abuzzahab et al., N Engl J Med, 349: 2211-2222 (2003)). Synthetic tyrosine kinase inhibitors (tyrphostins) have been described (Parrizas et al., Endocrinology, 138: 1427-1433 (1997)), including substrate-competitive inhibitors of IGF-1R kinase (Blum et al., Biochemistry, 39: 15705-15712 (2000)).

The cytoplasmic tyrosine kinase proteins are activated by the binding of the ligand to the extracellular domain of the receptor. After ligand binding, phosphorylated receptors recruit and phosphorylate docking proteins, including the insulin receptor substrate-1 protein family (IRS1), IRS2, Shc, Grb 10, and Gab1 (Avruch, Mol. Cell. Biochem., 182: 31-48 (1998); Tartare-Deckert et al., J. Biol. Chem., 270: 23456-23460 (1995); He et al., J. Biol. Chem. 271: 11641-11645 (1996); Dey et al., Mol. Endocrinol., 10: 631-641 (1996)); Peruzzi et al., J. Cancer Res. Clin. Oncol., 125:166-173 (1999); Dey et al., Mol. Endocrinol. 10: 631-641 (1996); Morrione et al., Cancer Res. 56: 3165-3167 (1996); Roth et al., Cold Spring Harbor Symp. Quant. Biol., 53: 537-543 (1988); White, Mol. Cell. Biochem., 182: 3-11 (1998); Laviola et al., J. Clin. Invest., 99: 830-837 (1997); Cheatham et al., Endocrin. Rev., 16: 117-142 (1995); Jackson et al., Oncogene, 20: 7318-7325 (2001); Nagle et al., Mol Cell Biol, 24: 9726-9735 (2004); Zhang et al., Breast Cancer Res. Treat., 83: 161-170 (2004)), leading to the activation of different intracellular mediators. IRS1 is the predominant signaling molecule activated by IGF-I, insulin, and interleukin-4 in estrogen receptor-positive human breast cancer cells (Jackson et al., J. Biol. Chem. 273: 9994-10003 (1998); Pete et al., Endocrinology, 140: 5478-5487 (1999)). The phosphatase PTP1D (syp) binds to IGF-1R, insulin receptor, and others (Rocchi et al., Endocrinology, 137: 4944-4952 (1996)). mSH2-B and vav are also binders of the IGF-1R (Wang and Riedel, J. Biol. Chem., 273: 3136-3139 (1998)).

The availability of substrates can dictate the final biological effect connected with the activation of IGF-1R. When IRS1 predominates, the cells tend to proliferate and transform. When Shc dominates, the cells tend to differentiate (Valentinis et al., J. Biol. Chem., 274: 12423-12430 (1999)). The route mainly involved in protection against apoptosis is via phosphatidyl-inositol 3-kinases (PI 3-kinases) (Prisco et al., Horm. Metab. Res., 31: 80-89 (1999)). IGF-1R and IRS1 can influence cell-cell interactions by modulating interaction between components of adherens junctions, including cadherin and beta-catenin (Playford et al Proc Nat Acad Sci (USA), 97: 12103-12108 (2000); Reiss et al., Oncogene, 19: 2687-2694 (2000)). See also Blakesley et al., In: The IGF System. Humana Press., 143-163 (1999)). Garrett et al., Nature, 394: 395-399 (1998) discloses the crystal structure of the first three domains of IGF-1R.

IGFs activate IGF-1R by triggering autophosphorylation of the receptor on tyrosine residues (Butler et al., Comparative Biochemistry and Physiology, 121:19 (1998)). IGF-I and IGF-II function both as endocrine hormones in the blood, where they are predominantly present in complexes with IGF binding proteins, and as paracrine and autocrine growth factors that are produced locally (Humbel, Eur. J. Biochem., 190, 445-462 (1990); Cohick and Clemmons, Annu. Rev. Physiol. 55: 131-153 (1993)). The domains of IGF-1R critical for its mitogenic, transforming, and anti-apoptotic activities have been identified by mutational analysis. For example, the tyrosine 1251 residue of IGF-1R has been found critical for anti-apoptotic and transformation activities but not for mitogenic activity (O'Connor et al., Mol. Cell. Biol., 17: 427-435 (1997); Miura et al., J. Biol. Chem., 270: 22639-22644 (1995)).

IGF binding proteins (IGFBPs) exert growth-inhibiting effects by, e.g., competitively binding IGFs and preventing their association with IGF-1R. The interactions among IGF-I, IGF-II, IGF-1R, acid-labile subunit (ALS), and IGFBPs affect many physiological and pathological processes such as development, growth, and metabolic regulation. See, e.g., Grimberg et al., J. Cell. Physiol., 183: 1-9 (2000). Six IGF binding proteins (IGFBPs) with specific binding affinities for the IGFs have been identified in serum (Yu and Rohan, J. Natl. Cancer Inst., 92: 1472-89 (2000)). See also U.S. Pat. No. 5,328,891; U.S. Pat. No. 5,258,287; EP 406272B1; and WO 89/09268. Only about 1% of serum IGF-I is present as free ligand; the remainder is associated with IGFBPs (Yu and Rohan, J. Natl. Cancer Inst., 92:1472-89 (2000)). References regarding the actions of IGFBPs, their variants, receptors, and inhibitors, including treating cancer, include US 2004/072776; US 2004/072285; US 2001/0034433; U.S. Pat. No. 5,200,509; U.S. Pat. No. 5,681,818; WO 2000/69454; U.S. Pat. No. 5,840,673; WO 2004/07543; US 2004/0005294; WO 2001/05435; WO 2000/50067; WO 2006/0122141; U.S. Pat. No. 7,071,160; and WO 2000/23469.

IGF-1R is homologous to insulin receptor (IR), having a sequence similarity of 84% in the beta-chain tyrosine-kinase domain and of 48% in the alpha-chain extracellular cysteine-rich domain (Ullrich et al., EMBO, 5: 2503-2512 (1986); Fujita-Yamaguchi et al., J. Biol. Chem., 261: 16727-16731 (1986)). IR is also described, e.g., in Vinten et al., Proc. Natl. Acad. Sci. USA, 88: 249-252 (1991); Belfiore et al., J. Biol. Chem., 277: 39684-39695 (2002); and Dumesic et al., J. Endocrin. Metab., 89: 3561-3566 (2004).

Although IR and IGF-1R similarly activate major signaling pathways, differences exist in recruiting certain docking proteins and intracellular mediators between the receptors (Sasaoka et al., Endocrinology, 137: 4427-34 (1996); Nakae et al., Endocrin. Rev., 22: 818-35 (2001); DuPont and LeRoith, Horm. Res., 55, Suppl. 2, 22-26 (2001); Koval et al., Biochem. J., 330: 923-32 (1998)). Thus, IGF-1R mediates mitogenic, differentiation, and anti-apoptosis effects, while activation of IR mainly involves effects at the metabolic pathways level (Baserga et al., Biochim. Biophys. Acta, 1332: F105-126 (1997); Baserga, Exp. Cell. Res., 253: 1-6 (1999); De Meyts et al., Ann. N.Y. Acad. Sci., 766: 388-401 (1995); Prisco et al., Horm. Metab. Res., 31: 80-89 (1999); Kido et al., J. Clin. Endocrinol. Metab., 86: 972-79 (2001)). Insulin binds with high affinity to IR (100-fold higher than to IGF-1R), while IGFs bind to IGF-1R with 100-fold higher affinity than to IR.

Because of their homology, these receptors can form hybrids containing one IR dimer and one IGF-1R dimer (Pandini et al., Cliff. Carte. Res., 5:1935-19 (1999); Soos et al., Biochem. J., 270, 383-390 (1990); Kasuya et al., Biochemistry, 32, 13531-13536 (1993); Seely et al., Endocrinology, 136: 1635-1641 (1995); Bailyes et al., Biochem. J., 327: 209-215 (1997); Federici et al., Mol. Cell. Endocrinol., 129: 121-126 (1997)). While both IR and IGF-1R were over-expressed in all breast cancer samples tested, hybrid receptor content consistently exceeded levels of both homo-receptors by approximately 3-fold (Pandini et al., Clin. Carc. Res. 5: 1935-44 (1999)). Although hybrid receptors are composed of IR and IGF-1R pairs, the hybrids bind selectively to IGFs, with affinity similar to that of IGF-1R, and only weakly bind insulin (Siddle and Soos, The IGF System. Humana Press, pp. 199-225 (1999)). Activation of IGF-1R mostly requires binding to ligand (Kozma and Weber, Mol. Cell. Biol., 10: 3626-3634 (1990)).

In liver, spleen, or placenta, hybrids are more represented than IGF-1R (Bailyes et al., supra). Breast tumoral cells specifically present on their surface IGF-1R, as well as IRs and many hybrids (Sciacca et al., Oncogene, 18: 2471-2479 (1999); Vella et al., Mol. Pathol., 54: 121-124 (2001)). Hybrids may also be overexpressed in thyroid and breast cancers (Belfiore et al., Biochimie (Paris) S1, 403-407 (1999)).

Two splice variants of IR have been reported. IR-B is the predominant IR isoform in normal adult tissues that are targets for the metabolic effects of insulin (Moller et al., Mol. Endocrinol., 3: 1263-1269 (1989); Mosthaf et al., EMBO J., 9: 2409-2413 (1990)). The IR isoform A variant is more often prevalent in cancer cells and fetal tissues (Frasca et al., Mol. Cell. Biol., 19: 3278-3288 (1999); DeChiara et al., Nature, 345: 78-80 (1990); Louvi et al., Dev. Biol., 189: 33-48 (1997); Pandini et al., J. Biol. Chem., 277: 39684-39695 (2002)).

The type II IGF receptor (IGF-IIR or mannose-6-phosphate (MOP) receptor) has high affinity for IGF-II, but lacks tyrosine kinase activity and does not apparently transmit an extracellular signal (Oases et al., Breast Cancer Res. Treat., 47: 269-281 (1998)). Because it results in the degradation of IGF-II, it is considered a sink for IGF-II, and its loss has been demonstrated in human cancer (MacDonald et al., Science, 239: 1134-1137 (1988)). Loss of IGF-IIR in tumor cells can enhance growth potential through release of its antagonistic effect on the binding of IGF-II with the IGF-IR (Byrd et al., J. Biol. Chem., 274: 24408-24416 (1999)).

Most normal tissues express IGF-1R (Werner et al., “The insulin-like growth factor receptor: molecular biology, heterogeneity, and regulation” In: Insulin-like Growth Factors: Molecular and Cellular Aspects, LeRoith (ed.) pp. 18-48 (1991)), which, e.g., promotes neuronal survival, maintains cardiac function, and stimulates bone formation and hematopoiesis (Zumkeller, Leuk. Lymphoma, 43: 487-491 (2002); Rosen, Best Pract Res Clin Endocrinol Metab, 18: 423-435 (2004); Leinninger and Feldman, Endocr Dev, 9: 135-159 (2005); Saetrum Opgaard and Wang, Growth Horm IGF Res, 15: 89-94 (2005); Wang et al., Mol Cancer Ther, 4: 1214-1221 (2005)). Also, disruption of IGF-1R affects survival of the pancreatic beta cells (Withers et al., Nat Genet, 23: 32-40 (1999)). See also LeRoith, Endocrinology, 141: 1287-1288 (2000) and LeRoith, New England J. Med., 336: 633-640 (1997).

IGF-1R has been considered to be quasi-obligatory for cell transformation (Adams et al., supra; Cohen et al., Clin. Cancer Res., 11: 2063-2073 (2005); Baserga, Oncogene, 19: 5574-5581 (2000)), and has been implicated in promoting growth, transformation, and survival of tumor cells (Blakesley et al., J. Endocr., 152: 339-344 (1997); Kaleko et al., Mol. Cell. Biol., 10: 464-473 (1990); Macaulay, supra; Baserga et al., Endocrine, 7: 99-102 (1997)). Several types of tumors are known to express higher than normal levels of IGF-1R (Khandwala et al., Endocrine Reviews, 21: 215-244 (2000); Werner and LeRoith, Adv. Cancer Res., 68: 183-223 (1996); Happerfield et al., J. Pathol., 183: 412-417 (1997); Frier et al., Gut, 44: 704-708 (1999); van Dam et al., J. Clin. Pathol., 47: 914-919 (1994); Xie et al., Cancer Res., 59: 3588-3591 (1999); Bergmann et al., Cancer Res., 55: 2007-2011 (1995)).

IGF-1R over-expression or elevated levels are shown, e.g., in human lung (Quinn et al., J. Biol. Chem., 271: 11477-11483 (1996); Kaiser et al., J. Cancer Res. Clin Oncol., 119: 665-668 (1993); Moody et al., Life Sciences, 52: 1161-1173 (1993); Macauley et al., Cancer Res., 50: 2511-2517 (1990)), ovary (Macaulay, Br. J. Cancer, 65: 311-320 (1990)), cervix (Steller et al., Cancer Res., 56: 1762 (1996)), breast (Ellis et al., Breast Cancer Res. Treat., 52:175 (1998); Cullen et al., Cancer Res., 50: 48-53 (1990); Gooch et al., Breast Cancer Res. Treat., 56:1-10 (1999); Webster et al., Cancer Res., 56: 2781 (1996); Pekonen et al., Cancer Res., 48: 1343 (1998); Peyrat and Bonneterre, Cancer Res., 22: 59-67 (1992); Lee and Yee, Biomed. Pharmacother., 49: 415-421 (1995); Turner et al., Cancer Research, 57: 3079-3083 (1997); Pollak et al., Cancer Lett., 38: 223-230 (1987); Pandini et al., Cancer Res., 5: 1935 (1999); Foekens et al., Cancer Res. 49: 7002-7009 (1989); Cullen et al., Cancer Res., 49: 7002-7009 (1990); Arteaga et al., J. Clin. Invest., 84: 1418-1423 (1989)), myeloma (Ge and Rudikoff, Blood, 96: 2856-2861 (2000)), sarcoma (van Valen et al., J. Cancer Res. Clin. Oncol., 118: 269-275 (1992); Xie et al., Cancer Res., 59: 3588 (1999); Scotlandi et al., Cancer Res., 56: 4570-4574 (1996)), prostate (Nickerson et al., Cancer Res., 61: 6276-6280 (2001); Chan et al., Science, 279:563 (1998); Hellawell et al., Cancer Res., 62: 2942-2950 (2002)), melanoma ((Hellawell et al., Cancer Res., 62: 2942-2950 (2002); All-Ericsson et al., Invest. Opthalmol. Vis. Sci., 43: 1-8 (2002)), and colon and colorectum (Hassan and Macaulay, Ann. Oncol., 13: 349-356 (2002); Weber et al., Cancer, 95: 2086-2095 (2002); Remaole-Bennet et al., J. Clin. Endocrinol. Metab., 75: 609-616 (1992); Guo et al., Gastroenterol., 102: 1101-1108 1992)). See also Goldring et al., Eukar. Gene Express., 1: 319-326 (1991).

Overexpression of human IGF-1R resulted in ligand-dependent anchorage-independent growth of NIH 3T3 or Rat-1 fibroblasts, and inoculation of these cells caused a rapid tumor formation in nude mice (Kaleko et al., Mol. Cell. Biol., 10: 464-473 (1990)). Soluble IGF-1R has been used to induce apoptosis in tumor cells in vivo and inhibit tumorigenesis in an experimental animal system (D'Ambrosio et al., Cancer Res. 56: 4013-4020 (1996)). See also Navarro and Baserga, Endocrinology, 142, 1073-1081 (2001).

Several reviews describe reasons for targeting the IGF system in cancer. See, for example, Pollak et al., Nat Rev Cancer, 4: 505-518 (2004); Yee, British J. Cancer, 94: 465-468 (2006); Bohula et al., Anti-Cancer Drugs, 14: 669-682 (2003); Surmacz, Oncogene, 22: 6589-97 (2003); Bahr and Groner, Growth Hormone and IGF Research 14: 287-295 (2004); Guillemard and Saragovi, Current Cancer Drug Targets, 4: 313-326 (2004); Jerome et al., Seminars in Oncology 31/1 Suppl. 3 (54-63) (2004); Zhang and Yee, Breast Disease, 17: 115-124 (2003); Samani and Brodt, Surgical Oncology Clinics of North America, 10: 289-312 (2001); Nahta et al., Oncologist, 8: 5-17 (2003); Dancey and Chen, Nature Reviews, 5: 649-659 (2006); Jones et al., Endocr. Relat. Cancer, 11:793-814 (2004); Schedin, Nature Reviews, 6: 281-290 (2006); Thorne and Lee, Breast Disease, 17: 105-114 (2003); Minchinton and Tannock, Nature Reviews, 6: 583-592 (2006); and Kurmasheva and Houghton, Biochim. Biophys. Acta, 1766: 1-22 (2006).

Epidemiological studies show a correlation of elevated plasma level of IGF-I with increased risk for prostate cancer, colon cancer, lung cancer, and breast cancer, including in humans (Chan et al., Science, 279: 563-566 (1998); Wolk et al., J. Natl. Cancer Inst., 90: 911-915 (1998); Ma et al., J. Natl. Cancer Inst., 91: 620-625 (1999); Yu et al., J. Natl. Cancer Inst., 91: 151-156 (1999); Pollak, Eur. J. Cancer 36:1224-1228 (2000); Wu et al., Cancer Res. 62: 1030-1035 (2002); Wu et al., Clin. Cancer Res., 11: 3065-3074 (2005); Renehan et al., Lancet, 363(9418): 1346-1353 (2004); Hankinson et al., Lancet, 351: 1393-1396 (1998)). Constitutive expression of IGF-I in epidermal basal cells of transgenic mice promotes spontaneous tumor formation (DiGiovanni et al., Cancer Res., 60: 1561-1570 (2000); Bol et al., Oncogene, 14: 1725-1734 (1997)). See also Pravtcheva and Wise, J Exp Zool, 281(1): 43-57 (1998) regarding studies showing that the IGF system can drive tumorigenesis in animal models. IGF-I and IGF-II have been shown in vitro to be potent mitogens for several human tumor cell lines such as lung cancer, breast cancer, colon cancer, osteosarcoma and cervical cancer (Ankrapp and Bevan, Cancer Res., 53: 3399-3404 (1993); Hermanto et al., Cell Growth& Differentiation, 11: 655-664 (2000); Guo et al., J. Am. Coll. Surg., 181: 145-154 (1995); Kappel et al., Cancer Res., 54: 2803-2807 (1994); Steller et al., Cancer Res., 56: 1761-1765 (1996)). Strategies are reported to prevent cancer by lowering plasma IGF-I levels or inhibiting IGF-1R function (e.g., Wu et al., Cancer Res., 62: 1030-1035 (2002); Grimberg and Cohen, J. Cell. Physiol., 183: 1-9 (2000)).

Over-expression of IGF-II in cell lines and tumors occurs with high frequency and may result from loss of genomic imprinting of the IGF-II gene (Yaginuma et al., Oncology, 54: 502-507 (1997)). Epigenetic changes (such as loss of imprinting at the IGF-II locus) frequently occurs in colon and ovarian cancers as well as in several pediatric malignancies (Feinberg, Semin Cancer Biol, 14: 427-432 (2004)). WO 2004/10850 discloses identifying loss of imprinting of the IGF-II gene in a subject by analyzing a biological sample for hypomethylation of a differentially methylated region (DMR) of the H19 gene and/or IGF-II gene.

In addition, metastatic cancer cells possess higher expression of IGF-II and IGF-1R than tumor cells less likely to metastasize (Guerra et al., Int. J. Cancer, 65: 812-820 (1996)). IGF-1R knockout-derived mouse embryo fibroblasts grow at significantly reduced rates in culture medium containing 10% serum and fail to be transformed by many oncogenes (Sell et al., Proc. Natl. Acad. Sci., USA, 90: 11217-11221 (1993); Sell et al., Mol. Cell. Biol., 14: 3604-3612 (1994); Morrione, Virol., 69: 5300-5303 (1995); Coppola et al., Mol. Cell. Biol., 14: 4588-4595 (1994); DeAngelis et al., J. Cell. Physiol., 164: 214-221 (1995)). Resistance to the HER-2 antibody HERCEPTIN® (trastuzumab) in some forms of breast cancer may be caused by activation of IGF-1R signaling (Nahta et al., Cancer Res, 65: 11118-11128 (2005); Lu et al., J. Natl. Cancer Inst. 93: 1852-1857 (2001)).

For reviews of how IGF-I/IGF-1R interaction mediates cell proliferation and plays a role in the growth of a variety of human tumors, see, e.g., Goldring et al., Eukar. Gene Express., 1:31-326 (1991) and Werner and LeRoith, Adv. Cancer Res. 68: 183-223 (1996). IGF-1R mechanisms and signaling are described, for example, in Datta et al., Genes and Development, 13: 2905-2927 (1999); Kulik et al., Mol. Cell. Biol. 17: 1595-1606 (1997); Dufourny et al., J. Biol. Chem., 272: 31163-31171 (1997); and Parrizas et al., J. Biol. Chem., 272: 154-161 (1997). See also Baserga, Expert Opin Ther Targets, 9: 753-768 (2005)).

Enhanced tyrosine phosphorylation of IGF-1R has been detected in human medulloblastoma (Del Valle et al., Clin. Cancer Res., 8: 1822-1830 (2002)) and in human breast cancer (Resnik et al., Cancer Res., 58: 1159-1164 (1998)). Deregulated expression of IGF-I in prostate epithelium leads to neoplasia in transgenic mice (DiGiovanni et al., Proc. Natl. Acad. Sci. USA, 97: 3455-3460 (2000)). Also, IGF-I appears to be an autocrine stimulator of human gliomas (Sandberg-Nordqvist et al., Cancer Res., 53: 2475-2478 (1993)), while IGF-I stimulated the growth of fibrosarcomas that overexpressed IGF-1R (Butler et al., Cancer Res., 58: 3021-3027 (1998)). Individuals with “high-normal” levels of IGF-I have an increased risk of common cancers compared to individuals with IGF-I levels in the “low-normal” range (Rosen et al., Trends Endocrinol. Metab., 10: 136-41 (1999)). Many of these tumor cell types respond to IGF-I with a proliferative signal in culture (Nakanishi et al., J. Clin. Invest., 82: 354-359 (1988); Freed et al., J. Mol. Endocrinol., 3: 509-514 (1989)), and autocrine or paracrine loops for proliferation in vivo have been suggested (Yee et al., Mol. Endocrinol., 3: 509-514 (1989); Yu and Rohan, J. Natl. Cancer Inst., 92: 1472-1489 (2000)).

IGF-1R activation can retard programmed cell death (Harrington et al., EMBO J., 13: 3286-3295 (1994); Sell et al., Cancer Res., 55: 303-305 (1995); Rodriguez-Tarduchy et al., J. Immunol., 149: 535-540 (1992); Singleton et al., Cancer Res., 56: 4522-4529 (1996)). Activated IGF-1R signals PI3K and downstream phosphorylation of Akt, or protein kinase B. Akt can block via phosphorylation molecules such as BAD that are essential for initiating programmed cell death and inhibit initiation of apoptosis (Datta et al., Cell, 91: 231-241 (1997)). The anti-apoptotic effect induced by the IGF-I/IGF-1R system correlates to chemo-resistance induction in various tumors (Grothey et al., J. Cancer Res. Clin. Oncol., 125: 166-173 (1999)).

Activation of IGF signaling can promote the formation of spontaneous tumors in a mouse transgenic model (DiGiovanni et al., Cancer Res., 60: 1561-1570 (2000)). IGF over-expression can rescue cells from chemotherapy-induced cell death and may be important in tumor cell drug resistance (Gooch et al., Breast Cancer Res. Treat., 56: 1-10 (1999)). Hence, modulation of the IGF signaling pathway has increased tumor cell sensitivity to chemotherapeutic agents (Benin et al., Clinical Cancer Res., 7: 1790-1797 (2001)).

A decrease in the level of IGF-1R below wild-type levels was also shown to cause massive apoptosis of tumor cells in vivo, using, e.g., anti-sense inhibition (Resnicoff et al., Cancer Res., 54: 2218-2222 (1994); Resnicoff et al., Cancer Res., 54: 4848-4850 (1994); Liu et al., Cancer Res., 58, 5432-5438 (1998); Chernicky et al., Cancer Gene Therapy, 7: 384-395 (2000), Sun et al., Cell research (China), 11: 107-115 (2001); Resnicoff et al., Cancer Res., 55: 2463-2469 (1995); Lee et al., Cancer Res., 56: 3038-3041 (1996); Muller et al., Int. J. Cancer, 77: 567-571 (1998); Shapiro et al., J. Clin. Invest., 94: 1235-1242 (1994); Resnicoff et al., Cancer Res., 55: 3739-3741 (1995); Trojan et al., Science, 259: 94-97 (1993); Kalebic et al., Cancer Res., 54: 5531-5534 (1994); Prager et al., Proc. Natl. Acad, Sci. USA, 91: 2181-2185 (1994); Burfeind et al., Proc. Natl. Acad. Sci. USA, 93: 7263-7268 (1996); Wraight et al., Nat. Biotech., 18: 521-526 (2000); Baserga, Cancer Res., 55: 249-252 (1995); and U.S. Pat. No. 6,340,674). Using the yeast two-hybrid system it was shown that p85, the regulatory domain of phosphatidyl inositol 3 kinase (PI3K), interacts with IGF-1R (Lamothe et al., FEBS Lett., 373: 51-55 (1995); Tartare-Decker et al., Endocrinology, 137: 1019-1024 (1996)). Another binding partner of IGF-1R, SHC, binds to other tyrosine kinases such as Trk, Met, EGF-R, and IR (Tartare-Deckert et al., J. Biol. Chem., 270: 23456-23460 (1995)). Downregulation of IGF-1R in mouse melanoma cells led to enhancement of radiosensitivity, reduced radiation-induced p53 accumulation and serine phosphorylation, and radioresistant DNA synthesis (Macaulay et al., Oncogene, 20: 4029-4040 (2001)). See also Wraight et al. (Nature Biotechnology, 18: 521-526 (2000)), showing reversal of epidermal hyperplasia in a mouse model of psoriasis using IGF-1R anti-sense oligonucleotides.

Transgenic mice overexpressing IGF-II specifically in the mammary gland develop mammary adenocarcinoma (Bates et al., Br. J. Cancer, 72: 1189-1193 (1995)), and transgenic mice overexpressing IGF-II under the control of a more general promoter develop more tumor types (Rogler et al., J. Biol. Chem., 269: 13779-13784 (1994)). At physiologic concentrations of insulin, breast cancer cells are stimulated to proliferate in vitro (Osborne et al., Proc Natl Acad Sci USA, 73: 4536-4540 (1976)). Activation of IR-A by IGF-II has been shown in breast cancer cell lines (Sciacca et al., supra). Hence, inhibition of both IGF-1R and IR may be required for optimal suppression of IGF signaling pathways.

Activation of the IGF system has been implicated in several pathologies besides cancer, including acromegaly and gigantism (Drange and Melmed. In: The IGF System. Humana Press., 699-720 (1999); Barkan, Cleveland Clin. J. Med., 65:343:347-349 (1998); Ben-Schlomo et al., Endocrin. Metab. Clin. North. Am., 30: 565-583 (2001)), atherosclerosis and smooth muscle restenosis of blood vessels following angioplasty (Bayes-Genis et al., Circ. Res., 86: 125-130 (2000)), diabetes or complications thereof, such as microvascular proliferation and retinal neovascularization (Smith et al., Nature Med., 12: 1390-95 (1999)), and psoriasis (Wraight et al., Nature Biotech., 18: 521-526 (2000)). Decreased IGF-I levels are associated with, e.g., small stature (Laron, Paediatr. Drugs, 1: 155-159 (1999)), neuropathy, decrease in muscle mass, and osteoporosis (Rosen et al., Trends Endocrinol. Metab., 10: 136-141 (1999)).

Calorie restriction has been reported to increase life span in a number of animal species, including mammals, and is additionally the most potent broadly acting cancer-prevention regimen in experimental carcinogenesis models. A key biological mechanism underlying many of its beneficial effects is the IGF-I pathway (Hursting et al., Annu. Rev. Med., 54:131-152 (2003). US 2006/0078533 discloses a method for prevention and treatment of aging and age-related disorders, including atherosclerosis, peripheral vascular disease, coronary artery disease, osteoporosis, type 2 diabetes, dementia, and some forms of arthritis and cancer in a subject using an effective dosage of, e.g., tyrosine kinase inhibitors/antibodies. EP 1808070 (Institute Pasteur) discloses a non-human animal as an experimental model for neurodegenerative diseases with an alteration in the biological activity of the IGF-1R found in the epithelial cells in the choroid plexus of the cerebral ventricles.

Using anti-sense and nucleic acids to antagonize IGF-1R is described, e.g., in Wraight et al., Nat. Biotech., 18: 521-526 (2000); U.S. Pat. No. 5,643,788; U.S. Pat. No. 6,340,674; US 2003/0031658; U.S. Pat. No. 6,340,674; U.S. Pat. No. 5,456,612; U.S. Pat. No. 5,643,788; U.S. Pat. No. 6,071,891; WO 2002/101002; CN 1237582A; CN 1117097B; WO 1999/23259; WO 2003/100059; US 2004/127446; US 2004/142895; US 2004/110296; US 2004/006035; US 2003/206887; US 2003/190635; US 2003/170891; US 2003/096769; U.S. Pat. No. 5,929,040; U.S. Pat. No. 6,284,741; US 2006/0234239; and U.S. Pat. No. 5,872,241.

Further, US 2005/0255493 discloses reducing IGF-1R expression by RNA interference using short double-stranded RNA.

In addition, inhibitory peptides targeting IGF-1R have been generated that possess anti-proliferative activity in vitro and in vivo (Pietrzkowski et al., Cancer Res., 52:6447-6451 (1992); Haylor et al., J. Am. Soc. Nephrol., 11:2027-2035 (2000)). Growth can also be inhibited using peptide analogues of IGF-I (Pietrzkowski et al., Cell Growth & Diff., 3: 199-205 (1992); Pietrzkowski et al., Mol. Cell. Biol., 12: 3883-3889 (1992)). In addition, dominant-negative mutants of IGF-1R (Li et al., J. Biol. Chem., 269: 32558-32564 (1994); Jiang et al., Oncogene, 18: 6071-6077 (1999); Scotlandi et al., Int. J. Cancer, 101: 11-16 (2002); Seely et al., BMC Cancer, 2: 15 (2002)) can reverse the transformed phenotype, inhibit tumorigenesis, and induce loss of the metastatic phenotype. A C-terminal peptide of IGF-1R has been shown to induce apoptosis and significantly inhibit tumor growth (Reiss et al., J: Cell. Phys., 181:124-135 (1999)). Also, a soluble form of IGF-1R inhibits tumor growth in vivo (D'Ambrosio et al., Cancer Res., 56: 4013-4020 (1996)).

Additional peptides that antagonize IGF-1R or treat cancer involving IGF-I include those described by U.S. Pat. No. 6,084,085; U.S. Pat. No. 5,942,489; WO 2001/72771; WO 2001/72119; US 2004/0086863; U.S. Pat. No. 5,633,263; and US 2003/0092631. See also U.S. Pat. No. 7,173,005 on peptide sequences capable of binding to insulin and/or IGF receptors with either agonist or antagonist activity. Moreover, the company Allostera is developing IGF-1R-directed peptides (Bioworld Today published May 19, 2006 (Vol. 17, page 1).

U.S. Pat. No. 7,020,563 discloses a method of designing agonists and antagonists to IGF-1R, by identifying compounds that modulate binding of a ligand to IGF-1R. This method comprises designing or screening for a compound that binds to the structure formed by amino acids having certain atomic coordinates, where binding of the compound to the structure is favored energetically, and testing the compound designed or screened for its ability to modulate binding of the ligand to IGF-1R in vivo or in vitro. U.S. Pat. No. 7,020,563 and EP 1,034,188 disclose identifying agonist and antagonist candidates to IGF-1R using its molecular structure. Selection of anti-cancer candidate compounds involving IGF-I or IGF-1R is described, e.g., in US 2004/0142381; US 2004/0121407; US 2003/0182668; U.S. Pat. No. 6,699,658 and U.S. Pat. No. 6,331,391.

Modified IGF-1R or IGF molecules are described, e.g., in WO 2003/80101; US 2004/0116335; U.S. Pat. No. 6,358,916; U.S. Pat. No. 6,610,302; U.S. Pat. No. 6,084,085; U.S. Pat. No. 5,942,412; U.S. Pat. No. 5,470,829; WO 2000/20023; U.S. Pat. No. 6,015,786; U.S. Pat. No. 6,025,332; U.S. Pat. No. 6,025,368; U.S. Pat. No. 6,514,937; U.S. Pat. No. 6,518,238; WO 2000/53219; and JP 5199878. Further, US 2006/0040358 and U.S. Pat. No. 6,913,883 report IGF-1R-interacting proteins.

Combination therapies involving IGF-1R inhibitors or IGF-I are described, e.g., in US 2004/0072760; US 2004/209930; WO 2004/030627; US 2004/0106605; WO 1993/21939; U.S. Pat. No. 5,731,325; US 2005/043233; US 2005/075358; WO 2005/041865; and U.S. Pat. No. 6,140,346. US 2006/0258569 discloses a method of treating cancer involving administering an IGF-1R agonist and a chemotherapeutic agent, as well as compounds for treating cancer comprising an IGF-1R ligand or IR ligand coupled to a chemotherapeutic agent. Additionally, EP 1,671,647 discloses a medicament for treating cancer in which a cancer therapeutic effect is synergistically increased using a substance inhibiting activities of IGF-I and IGF-II. IGF-1R inhibitors are useful to treat cancer (e.g., US 2004/0044203), as either single agents or with other anti-cancer agents (Burtrum et al., Cancer Research, 63: 8912-8921 (2003)). Also, US 2006/0193772 describes inhibitors of IGF-I and IGF-II to treat cancer.

Cancer vaccines involving IGF-I are described, e.g., in U.S. Pat. No. 5,919,459; EP 702563B1; WO 1994/27635; EP 1284144A1; WO 2003/015813; U.S. Pat. No. 6,420,172; EP 637201A4; and WO 1993/20691.

Small-molecule inhibitors to IGF-1R are described, e.g., in Garcia-Echeverria et al., Cancer Cell, 5: 231-239 (2004); Mitsiades et al., Cancer Cell, 5: 221-230 (2004); and Carboni et al., Cancer Res, 65: 3781-3787 (2005). Further, compounds have been developed that disrupt receptor activation, such as, for example, Vasilcanu et al., Oncogene, 23: 7854-7862 (2004), which describes a cyclolignan, picropodophyllin, which appears to be specific for IGF-1R (Girnita et al., Cancer Res, 64: 236-242 (2004); Stromberg et al., Blood, 107: 669-678 (2006)). Nordihydroguaiaretic acid (NDGA) also disrupts IGF-1R function (Youngren et al., Breast Cancer Res Treat, 94: 37-46 (2005)). Further examples of disclosures on such small-molecule inhibitors include WO 2002/102804; WO 2002/102805; WO 2004/55022; U.S. Pat. No. 6,037,332; WO 2003/48133; US 2004/053931; US 2003/125370; U.S. Pat. No. 6,599,902; U.S. Pat. No. 6,117,880; WO 2003/35619; WO 2003/35614; WO 2003/35616; WO 2003/35615; WO 1998/48831; U.S. Pat. No. 6,337,338; US 2003/0064482; U.S. Pat. No. 6,475,486; U.S. Pat. No. 6,610,299; U.S. Pat. No. 5,561,119; WO 2006/080450; WO 2006/094600; and WO 2004/093781 See also WO 2007/099171 (bicyclo-pyrazole inhibitors) and WO 2007/099166 (pyrazolo-pyridine derivative inhibitors). See also (Hubbard et al., AACR-NCI-EORTC Int Conf Mol Targets Cancer Ther (October 22-26, San Francisco) 2007, Abst A227) on Abbott Corporation's molecule A-928605.

Diagnostics involving IGF or IGF-1R are described in, e.g., US 2003/0044860; U.S. Pat. No. 6,410,335; US 2001/0018190 U.S. Pat. No. 6,645,770; U.S. Pat. No. 6,410,335; U.S. Pat. No. 6,448,086; WO 2001/53837; WO 2004/65583; WO 2001/25790; and WO 2002/31500. WO 2006/060419 and US 2006/0140960 disclose certain biomarkers for pre-selection of patients for anti-IGF-1R therapy. US 2007/190583 reports use of various biomarkers for cancer (including TGF-α, pS6, and IGF-1R) to assess a subject's suitability for treatment with an EGFR/ErbB2 kinase inhibitor such as lapatinib. U.S. Pat. No. 5,442,043 describes detecting IGF-1R on tumors.

WO 2002/17951 describes treatment of brain cancer with an IGF-I structural analog such as des-IGF; US 2003/0017146; U.S. Pat. No. 5,851,985; and U.S. Pat. No. 6,261,557 describe treatment of amino-acid deprived cancer patients with IGF-I optionally with arginine-decomposing enzyme; WO 1993/09816 describes a conjugate of IGF-I and radionucleotide to treat cancer; and WO 200413177 discloses use of mannose-6-phosphate/insulin-like growth factor-2 receptor (CD222) as regulator of urokinase plasminogen activator functions, useful for treating arteriosclerosis, restenosis, autoimmunity, inflammation and cancer.

Several antibodies, small molecules, and anti-sense molecules against IGF-1R have shown promise in mouse tumor models with little or no toxicity (Garber et al., J. Natl. Cancer Inst., 97: 790-92 (2005). Gualberto et al., “Inhibition of the insulin like growth factor 1 receptor by a specific monoclonal antibody in multiple myeloma”, J. Clin. Oncology, 41st Annual Meeting of the American-Society-of-Clinical-Oncology (May 13-17, 2005, Orlando, Fla. (published Jun. 1, 2005, vol. 23 (16): 1 Supp 203S, states that a biomarker assay was generated to support the clinical development of the anti-IGF-1R antibody CP-751,871. Flow cytometry of granulocytes was found to be a reliable biomarker of the activity of this antibody, and may contribute to define a therapeutic dose and regimen. Further, this antibody was found to effectively downregulate IGF-1R expression on peripheral blood leucocytes (PBLs).

Because small-molecule inhibitors of the IGF-1R kinase, however, often cross-inhibit the insulin receptor, antibody-based approaches afford better selectivity toward IGF-1R. In addition, unlike small-molecule agents, antibodies are not likely to cross the blood-brain barrier (Rubenstein et al., Blood, 101(2): 466-268 (2003)), reducing the risk of possible interference with the central nervous system. This is particularly relevant to cognitive function, because IGF-I has been suggested to be required for optimal performance of memory and learning throughout life (Sonntag et al., Ageing Res Rev, 4: 195-212 (2005)).

Antibodies to various growth-factor receptors and their ligands are known. For example, antibodies to EGF receptor are reported, e.g., in U.S. Pat. No. 5,891,996 and U.S. Pat. No. 7,060,808. Antibodies to IGF are described, e.g., in EP 1,505,075; EP 656,908B1; US 2006/0240015; WO 1994/04569; US 2006/0165695; EP 1,676,862; and EP 1,671,647. See also Feng et al., “Novel human monoclonal antibodies to insulin-like growth factor (IGF)-II that potently inhibit the IGF receptor type I signal transduction function,” Mol Cancer Ther., 5 (1):114-120 (2006) and US 2007196376 on antibodies to IGF-II.

Antibodies to IGF-1R, e.g., a mouse IgG1 monoclonal antibody designated αIR3 (Kull et al., J. Biol. Chem., 258:6561-6566 (1983); Arteaga and Osborne, Cancer Research, 49:6237-6241 (1989)), inhibit proliferation of many tumor cell lines (Arteaga et al., Breast Cancer Res. Treat., 22:101-106 (1992); Rohlik et al., Biochem. Biophys. Res. Commun., 149: 276-281 (1987); Arteaga et al., J. Clin. Invest., 84:1418-1423 (1989)). αIR3 is commonly used for IGF-1R studies in vitro, and exhibits agonistic activity in transfected 3T3 and CHO cells expressing human IGF-1R (Kato et al., J. Biol. Chem., 268:2655-2661 (1993); Steele-Perkins and Roth, Biochem. Biophys. Res. Commun., 171:1244-1251 (1990)). The binding epitope of αIR3 is inferred from chimeric insulin-IGF-I receptor constructs to be the 223-274 region of IGF-1R (Gustafson and Rutter, J. Biol. Chem., 265:18663-18667 (1990)). In MCF-7 human breast cancer cells (Dufourny et al., J. Biol. Chem., 272:31163-31171 (1997)), αIR3 incompletely blocks the stimulatory effect of exogenously added IGF-I and IGF-II in serum-free conditions by approximately 80%. Also, αIR3 does not significantly inhibit (less than 25%) the growth of MCF-7 cells in 10% serum (Cullen et al., Cancer Res., 50:48-53 (1990)).

Additional mouse monoclonal antibodies that inhibit IGF-1R activity include 1H7 (Li et al., Biochem. Biophys. Res. Comm., 196: 92-98 (1993); Xiong et al., Proc. Natl. Acad. Sci., U.S.A., 89: 5356-5360 (1992)) and MAB391 (R&D Systems; Minneapolis, Minn.). See also Zia et al., J. Cell. Biol., 24:269-275 (1996) regarding mouse monoclonal antibodies. Further, single-chain antibodies against IGF-1R have been shown to inhibit growth of MCF-7 cells in xenografts models (Li et al., Cancer Immunol. Immunother., 49: 243-252 (2000)) and to lead to down-regulation of cell-surface receptors (Sachdev et al., Cancer Res, 63: 627-635 (2003)).

Antibodies directed against human IGF-1R have also been shown to inhibit tumor-cell proliferation in vitro and tumorigenesis in vivo including cell lines derived from Ewing's osteosarcoma (Scotlandi et al., Cancer Res., 58:4127-4131 (1998)) and melanoma (Furlanetto et al., Cancer Res., 53:2522-2526 (1993)). See also Park and Smolen. In: Advances in Protein Chemistry. Academic Press. pp: 360-421 (2001); Thompson et al., Pediat. Res., 32: 455-459 (1988); Tappy et al., Diabetes, 37: 1708-1714 (1988); Weightman et al., Autoimmunity, 16:251-257 (1993); and Drexhage et al., Nether. J. of Med., 45:285-293 (1994).

Other publications on IGF-1R antibodies and their anti-tumor effects include, e.g., Benini et al., Clin. Cancer Res., 7: 1790-1797 (2001); Scotlandi et al., Cancer Gene Ther., 9: 296-307 (2002); Scotlandi et al., Int. J. Cancer, 101: 11-16 (2002); Brunetti et al., Biochem. Biophys. Res. Commun., 165: 212-218 (1989); Prigent et al., J. Biol. Chem., 265: 9970-9977 (1990); Pessino et al., Biochem. Biophys. Res. Commun., 162: 1236-1243 (1989); Surinya et al., J. Biol. Chem., 277: 16718-16725 (2002); Soos et al., J. Biol. Chem., 267: 12955-12963 (1992); Soos et al., Proc. Natl. Acad. Sci. USA, 86: 5217-5221 (1989); O'Brien et al., EMBO J., 6: 4003-4010 (1987); Taylor et al., Biochem. J., 242: 123-129 (1987); Soos et al., Biochem. J., 235: 199-208 (1986); Li et al., Biochem. Biophys. Res. Commun., 196: 92-98 (1993); Delafontaine et al., J. Mol. Cell. Cardiol., 26: 1659-1673 (1994); Morgan and Roth, Biochemistry, 25: 1364-1371 (1986); Forsayeth et al., Proc. Natl. Acad. Sci. USA, 84: 3448-3451 (1987); Schaefer et al., J. Biol. Chem., 265: 13248-13253 (1990); Hoyne et al., FEBS Lett., 469: 57-60 (2000); Tulloch et al., J. Struct. Biol., 125: 11-18 (1999); Dricu et al., Glycobiology, 9: 571-579 (1999); Kanter-Lewensohn et al., Melanoma Res., 8: 389-397 (1998); Hailey et al., Mol. Cancer. Ther., 1: 1349-1353 (2002); Maloney et al., Cancer Res, 63: 5073-5083 (2003); Goetsch et al., Int J Cancer, 113: 316-328 (2005); and Wang et al., supra). The monoclonal antibody binding sometimes results in endosomal degradation of the receptor (Sachdev et al., supra; Wang et al., supra).

Antibodies, nanobodies, and antibody-like molecules targeting growth factor receptors and receptor protein tyrosine kinases, including IGF-1R, and their various uses, including treating cancer, are described also in, e.g., US 2001/0005747; U.S. Pat. No. 5,833,985; EP 749325B1; WO 1995/24220; WO 2002/053596; WO 2004/083248; WO 2005/005635; US 2003/0165502; US 2002/0009739; US 2003/0158109; WO 2000/022130; WO 2007/000328; US 2003/0235582; US 2004/0265307; US 2005/186203; WO 2005/061541; US 2006/0233810; WO 2006/113483; US 2005/0249728; US 2004/0018191; US 2007/0059241; US 2007/0059305 U.S. Pat. No. 7,037,498; US 2005/244408; US 2005/281812; US 2004/0116330; US 2004/0202651; US 2004/0202655; US 2004/0228859; US 2005/0008642; US 2005/0069539; WO 2005/016967; US 2005/0084906; U.S. Pat. No. 7,241,444; WO 2007/092453; WO 2007/115814; WO 2007/115813; US 2007/0248600; US 2007/0243194; US 2005/0249730; WO 2003/59951; WO 2005/058967; WO 2002/05359; WO 2003/100008; WO 2003/106621; WO 2006/013472; US 2005/0136063; US 2005/048050; WO 2002/102973; WO 2002/102972; WO 2002/102854; WO 2004/87756; WO 2005/016967; U.S. Pat. No. 7,217,796; WO 2005/016970; WO 2005/082415; US 2006/0018910; US 2005/0281814; WO 2006/069202; WO 2007/00328; WO 2007/042289; WO 2007/093008; U.S. Pat. No. 6,524,832; WO 2007/012614; and US 2007/0099847. US 2004/0213792 discloses inhibiting cellular activation by IGF-I by administering an antagonist inhibiting binding of IAP to SHPS-1). WO 2007/095337 discloses an antibody-buffer formulation, including antibodies to receptors, and WO 2007/110339 discloses a formulation of IGF-1R monoclonal antibodies.

The insulin-like growth factor (IGF) signaling pathway is a major regulator of cellular proliferation, stress responses, apoptosis and transformation in mammalian cells that is dysregulated and activated in a wide range of human cancers. The central components of this signaling module are the IGF-1 receptor (IGF-1R), a homodimeric receptor tyrosine kinase, and its ligands IGF-I and IGF-II. Numerous studies have shown that ligand mediated stimulation of IGF-1R results in receptor clustering and autophosphorylation followed by transphosphorylation of the beta subunits (Hernandez-Sanchez et al., The Journal of Biological Chemistry 270(49):29176-29181 (December 1995)). These phosphorylation events create multiple docking sites for the substrate adaptor proteins IRS1, IRS2 and SHC, which are essential transducers and amplifiers of IGF-1R signaling that recruit signaling complexes to the membrane and result in proliferative and anti-apoptotic cellular responses (Baserga et al. Endocrine 7(1):99-102 (August 1997)). Mechanistic studies have shown that phosphorylation of IRS1 triggers activation of the PI3 kinase/Akt pathway and ultimately leads to sequestration and inhibition of the pro-apoptotic protein BAD as well as activation of the cell cycle initiator Cyclin D (Surmacz, E., Journal of Mammary Gland Biology and Neoplasia 5(1):95-105 (January 2000)), suggesting that inhibition of IGF-1R signaling may have both pro-apoptotic and anti-proliferative consequences.

Alterations of key components of IGF-1R signaling have also been shown to be associated with increased risk of cancer as well as neoplastic transformation. Specifically, high levels of circulating IGF-I have been shown to be associated with increased risk of developing breast, prostate, and colorectal cancer (Furstenberger et al., The Lancet Oncology 3(5):298-302 (May 2002)), while epigenetic loss of imprinting at the IGF-II locus has been shown to be common in colorectal cancer and to constitute a potential biomarker of colorectal cancer risk (Cui et al., Science 299(5613):1753-1755 (March 2003)). In addition, genetic studies have shown that overexpression of IGF-I leads to neoplastic transformation in prostate epithelium (Wilker et al., Molecular Carcinogenesis 25(2):122-131 (June 1999)), while overexpression of IGF-II in transgenic mice results in metastasizing mammary carcinomas, suggesting that these ligands can be key drivers of tumorigenesis when dysregulated and overexpressed (Pravtcheva and Wise, The Journal of Experimental Zoology 281(1):43-57 (May 1998)). A number of studies have suggested that IGF-1R expression is absolutely required for the acquisition and maintenance of a transformed phenotype in diverse genetic backgrounds and multiple cell types in vivo and in vitro (Baserga R., Cancer Research 55(2):249-252 (January 1995); Coppola et al., Molecular and Cellular Biology 14(7):4588-4595 (July 1994); Sell et al., PNAS 90(23):11217-11221 (December 1993)). Taken together, the role of IGF ligands in driving neoplastic transformation and the requirement of receptor activity for maintaining the transformed phenotype have implicated the IGF axis as an attractive candidate pathway for therapeutic intervention.

Indeed, by one recent estimate >25 molecules aimed at targeting IGF-1R as an anti-cancer therapy are currently in different stages of clinical and preclinical development at various pharmaceutical and biotechnology companies (Rodon et al., Molecular Cancer Therapeutics 7(9):2575-2588 (September 2008)). The two predominant strategies to target IGF-1R are specific kinase inhibitors or monoclonal antibodies raised against IGF-1R that can block receptor function. A key distinction between small molecule inhibitors and blocking antibodies is specificity, since IGF-1R is 84% identical to insulin receptor in the kinase domain and hence it is exceedingly difficult to design ATP mimetic kinase inhibitors that are selective only for IGF-1R. In contrast, antibodies that recognize specific epitopes unique to IGF-1R may be expected to have enhanced selectivity for IGF-1R, which could mitigate off-target toxicities that may result from inhibition of insulin receptor.

Development of a humanized, affinity matured anti-human IGF-1R monoclonal antibody, h10H5, has been previously described. Shang et al., Molecular Cancer Therapeutics 7(9):2599-2608 (September 2008); US 2009-0068110-A1. The antibody has been shown to have anti-tumor activity in mouse xenograft models and potently decreases Akt signaling as well as glucose uptake in preclinical models. The mechanism of action of h10H5 is similar to other blocking antibodies and involves blockade of ligand binding, cell surface downregulation of receptor levels, and downregulation of intracellular signaling mediated by Akt (Shang et al. supra). While h10H5 is effective in inhibiting in vitro proliferation of many types of tumor cells, it lacks activity in others. Therefore, an important outstanding question in the clinical development of agents such as h10H5 is whether predictive diagnostic tests can be developed to identify appropriate patient populations, allowing specific treatment of patients whose tumors show addiction to this pathway for continued survival and proliferation. Previous studies have examined the role of role of IGF-IR number in IGF-1-mediated mitogenesis and transformation of mouse embryo fibroblasts, in which a 3T3-cell derivative with targeted knockout of IGF-1R was transfected with an IGF-1R expression construct to generate clones expressing differing levels of IGF-1R (Rubini et al., Experimental Cell Research 230(2):284-292 (February 1997)).

Studies using Xenopus oocytes, which possess endogenous IGF-I receptors but have little or no IRS1, showed that microinjection of IRS1 protein resulted in a maturation response in direct proportion to the levels of injected IRS1 and suggested that IRS activity is necessary for the cellular response to IGF in this system (Chuang et al., PNAS 90(11):5172-5175 (June 1993)). In addition, previous studies in T47D-YA breast cancer cells suggested that IRS1 and IRS2 expression is required for proliferative and motility responses to IGF-1R activation in these cells, since in the absence of expression of either adaptor molecule IGF-IR activation was unable to stimulate proliferation or motility in T47D-YA cells but proliferative and motility responses were restored upon expression of IRS1 and IRS2, respectively (Byron et al., British Journal Of Cancer 95(9):1220-1228 (November 2006)).

A recent report has shown that IGF-1R expression can be detected on circulating tumor cells (CTCs) in hormone refractory prostate cancer and that levels of IGF-1R positive CTCs might have utility as a pharmacodynamic biomarker of response to the anti-IGF-1R targeting antibody CP-751,871 (de Bono et al., Clinical Cancer Research 13(12):3611-3616 (June 2007)).

Previous studies have suggested that IGF-1R levels are strongly associated with preclinical response to a humanized anti-IGF-1R antibody in Rhabdomyosarcoma cells and thus that levels of the target itself may constitute a predictive biomarker for response to IGF-1R targeting antibodies in this indication (Cao et al., Cancer Research 68(19):8039-8048 (October 2008)). Others have looked at the predictive value of phosphorylation of IGF-1R itself or of the substrate IRS1 as markers of pathway activation that may predict response, or at gene expression signature predictors of response to the small molecule inhibitor BMS-536924 (Rodon et al. supra). These studies have provided interesting hypotheses that await clinical validation, but as yet studies looking broadly at response in other tumor types where IGF-1R may play an important role, such as breast and colorectal cancer, have not been reported.

Gualberto et al. studied figitumumab (CP-751,871), a human IgG2 antibody, in non-small cell lung cancer (NSCLC) and concluded that IGF-1R and IGF-1 constitute independent mechanisms of sensitivity to figitumumab in NSCLC, and that determination of IGF-1R and epithelial-to-mesenchymal transition (EMT) markers may contribute to the identification of patients who could benefit from figitumumab therapy. Gualberto et al. “Molecular Basis for Sensivity to Figitumumab (CP-751,871) in Non-Small Cell Lung Cancer” Abstract 8091, ASCO 2009. Hixon et al. report that determining baseline levels of free IGF-1 may contribute to the identification of patients with NSCLC. Hixon et al. “Plasma Levels of Free Insulin Like Growth Factor 1 Predict the Clinical Benefit of Figitumumab (CP-751,871) in Non-Small Cell Lung Cancer” Abstract 3539, ASCO 2009.

### SUMMARY OF THE INVENTION

The insulin-like growth factor receptor (IGF-1R) pathway is required for the maintenance of the transformed phenotype in neoplastic cells and hence has been the subject of intensive drug discovery efforts. A key aspect of successful clinical development of targeted therapies directed against IGF-1R involves identification of responsive patient populations. Towards that end, experimental data is provided in the present application which identifies predictive biomarkers of response to an anti-IGF-1R targeting monoclonal antibody in breast and colorectal cancer. The data shows that levels of the IGF-1R receptor itself may have predictive value in these tumor types and identifies other gene expression predictors of in vitro response. Studies in breast cancer models suggest that IGF-1R expression is both correlated and functionally linked with estrogen receptor signaling, and provide a basis for both patient stratification and rational combination therapy with anti-estrogen targeting agents. In addition, the data indicates that levels of other components of the signaling pathway such as the adaptor proteins IRS1 and IRS2, as well as the ligand IGF-II, have predictive value.

With these data in mind, in a first aspect, the invention herein provides a method of treating cancer in a human patient comprising administering an IGF-1R inhibitor to the patient, provided the patient's cancer has been shown to express, at a level above the median for the type of cancer being treated, two or more biomarkers selected from the group consisting of IGF-1R, IGF-II, IRS1 and IRS2. Preferably the cancer is breast or colorectal cancer. Preferably the IGF-1R inhibitor is a human or humanized antibody that binds IGF-1R.

In another aspect, the invention provides a method of treating breast cancer in a human patient comprising administering an IGF-1R inhibitor to the patient, provided the patient's cancer has not been found to express IGF-1R at a level below the median for breast cancer.

The invention also concerns a method of treating breast cancer in a human patient comprising administering an IGF-1R inhibitor to the patient, provided the patient has been shown to express one or more biomarkers selected from the group consisting of IGF-1R, IRS1, IRS2, IGF-II, and estrogen receptor, at level above the median for breast cancer.

Also provided is a method of treating breast cancer in a human patient comprising administering a combination of an IGF-1R inhibitor and an estrogen inhibitor, wherein the combination results in a synergistic effect in the patient.

The invention, in another aspect, concerns a method for treating a patient with colorectal cancer, comprising administering a therapeutically effective amount of an IGF-1R inhibitor to the patient, provided the patient's cancer expresses IGF-1R at a level greater than the median level for IGF-1R expression in colorectal cancer.

The invention additionally provides a method for treating a patient with colorectal cancer, comprising administering a therapeutically effective amount of an IGF-1R inhibitor to the patient, provided the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1. In one embodiment, the patient's cancer further expresses IGF-1R at a level above the median for colorectal cancer.

Also provided is a method for selecting a therapy for a patient with cancer, comprising administering a therapeutically effective amount of an IGF-1R inhibitor to the patient, if the patient's cancer: has been shown to express, at a level above the median for the type of cancer being treated, two or more biomarkers selected from the group consisting of: IGF-1R, IGF-II, IRS1 and IRS2.

The invention further concerns a method for selecting a therapy for a patient with breast cancer, comprising administering a therapeutically effective amount of an IGF-1R inhibitor to the patient, provided the patient's cancer:

(a) has not been found to express IGF-1R at a level below the median for breast cancer; or
(b) has shown to express one or more biomarkers selected from the group consisting of IGF-1R, IRS1, IRS2, IGF-II, and estrogen receptor, at level above the median for breast cancer.

In addition, the invention concerns a method for selecting a therapy for a patient with colorectal cancer, comprising administering a therapeutically effective amount of an IGF-1R inhibitor to the patient, provided the patient's cancer:

(a) expresses IGF-1R at a level greater than the median level for IGF-1R expression in colorectal cancer; or
(b) expresses one or more biomarkers selected from the group consisting of: TOB1, CD24, MAP2K6, SMAD6, TNFSF10, PMP22, CTSL1, ZMYM2, PALM2, ICAM1, and GBE1.

In a further aspect, the invention concerns an article of manufacture comprising, packaged together, a pharmaceutical composition comprising an IGF-1R inhibitor in a pharmaceutically acceptable carrier and a package insert stating that the inhibitor or pharmaceutical composition is indicated for treating:

(a) a patient with cancer, if the patient's cancer has been shown to express, at a level above the median for the type of cancer being treated, two or more biomarkers selected from the group consisting of: IGF-1R, IGF-II, IRS1 and IRS2;
(b) a patient with breast cancer, if the patient's cancer has not been found to express IGF-1R at a level below the median for breast cancer;
(c) a patient with breast cancer, if the patient's cancer has shown to express one or more biomarkers selected from the group consisting of IGF-1R, IRS1, IRS2, IGF-II, and estrogen receptor, at level above the median for breast cancer;
(d) a patient with colorectal cancer, if patient's cancer expresses IGF-1R at a level greater than the median level for IGF-1R expression in colorectal cancer; or
(e) a patient with colorectal cancer, if the patient's cancer expresses one or more biomarkers selected from the group consisting of: TOB 1, CD24, MAP2K6, SMAD6, TNFSF10, PMP22, CTSL1, ZMYM2, PALM2, ICAM1, and GBE1.

Moreover, the invention provides a method for manufacturing an IGF-1R inhibitor or a pharmaceutical composition thereof comprising combining in a package the inhibitor or pharmaceutical composition and a package insert stating that the inhibitor or pharmaceutical composition is indicated for treating:

(a) a patient with cancer, if the patient's cancer has been shown to express, at a level above the median for the type of cancer being treated, two or more biomarkers selected from the group consisting of: IGF-1R, IGF-II, IRS1 and IRS2;
(b) a patient with breast cancer, if the patient's cancer has not been found to express IGF-1R at a level below the median for breast cancer;
(c) a patient with breast cancer, if the patient's cancer has shown to express one or more biomarkers selected from the group consisting of IGF-1R, IRS1, IRS2, IGF-II, and estrogen receptor, at level above the median for breast cancer;
(d) a patient with colorectal cancer, if patient's cancer expresses IGF-1R at a level greater than the median level for IGF-1R expression in colorectal cancer; or
(e) a patient with colorectal cancer, if the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1.

In addition, the invention provides a method for advertising an IGF-1R inhibitor or a pharmaceutically acceptable composition thereof comprising promoting, to a target audience, the use of the inhibitor or pharmaceutical composition thereof for treating:

(a) a patient with cancer, if the patient's cancer has been shown to express, at a level above the median for the type of cancer being treated, two or more biomarkers selected from the group consisting of: IGF-1R, IGF-II, IRS1 and IRS2;
(b) a patient with breast cancer, if the patient's cancer has not been found to express IGF-1R at a level below the median for breast cancer;
(c) a patient with breast cancer, if the patient's cancer has shown to express one or more biomarkers selected from the group consisting of IGF-1R, IRS1, IRS2, IGF-II, and estrogen receptor, at level above the median for breast cancer;
(d) a patient with colorectal cancer, if patient's cancer expresses IGF-1R at a level greater than the median level for IGF-1R expression in colorectal cancer; or
(e) a patient with colorectal cancer, if the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1.

### BRIEF DESCRIPTION OF THE DRAWINGS

FIGS. 1A-1C depict association of IGF-1R levels with h10H5 response and ER Status. In FIG. 1A, forty one breast cancer cell lines were screened for in vitro sensitivity to h10H5 using an ATP based cell viability assay. The left axis and bar chart shows IGF-1R mRNA level for each cell line as determined by gene expression microarray and the right axis and diamonds show the EC₅₀ for h10H5 in each cell line. The chart at the bottom shows estrogen receptor (ER) status for each cell line as determined by immunohistochemistry on a cell pellet tissue microarray. In FIG. 1B, a combination of high expression of IGF-1R and the substrates IRS1 and IRS2 is associated with in vitro response to h10H5 in breast cancer cells. Heatmap shows expression of IGF-1R, IGF-II and the substrates IRS1 and IRS2 in breast cancer cell lines. Color coding is by z-scores and red indicates high expression (2 standard deviations (SD) above the mean) and green low expression (2 SD below mean). Purple indicates cell lines that are sensitive to h10H5 and yellow lines that are insensitive. In FIG. 1C, pharmacodynamic response of sensitive MCF-7 and insensitive MDA-MB-231 cells to h10H5 treatment. Cells were treated with 1 mg/mL h10H5 for 24 hours and lysates used for immunoblotting with antibodies detecting the epitopes indicated to the right of the figure.

FIGS. 2A-2D depict combined effects of ER and IGF-1R targeting in vitro and in vivo. In FIG. 2A, expression of IGF-1R and IGF-I in estrogen receptor high and low human breast tumors and protein expression in ER+ tumors is shown. Heat map shows expression determined by Affymetrix microarray and is color coded by z-scores. In FIG. 2B, affect of siRNA ablation of ESR1, the gene encoding estrogen receptor, or IGF-1R siRNA ablation on mRNA levels of ESR1 and IGF-1R in MCF-7 breast cancer cells is shown. Cell were transfected with a control siRNA (NTC) or siRNAs targeting ESR1 or IGF-1R for 72 hours, RNA was prepared and IGF-1R levels assesses by qRT-PCR. IGF-1R is knocked down by IGF-1R siRNA treatment and also substantially reduced by ESR1 depletion. IGFBP2 is shown as a control to demonstrate that not all pathway components are downregulated by ESR1 and IGF-1R treatment. In FIG. 2C, shows effects of combined in vitro targeting of estrogen receptor with the selective inhibitor Faslodex and IGF-1R with h10H5. Cells were cultured in 2.5% FBS. Trastuzumab is included as an antibody control since MCF-7 cells are HER2 negative and do not show any response to anti-HER2 targeting agents. The combination of Faslodex and h10H5 shows substantially greater inhibition of cell viability than either single agent. FIG. 2D shows combined treatment with tamoxifen and h10H5 shows superior tumor growth inhibition to either single agent in xenografted MCF-7 tumors. Exogenous estrogen was provided in drinking water. h10H5 was administered weekly as indicated by the arrowheads and a tamoxifen slow release pellet was implanted at the start of the study (arrow).

FIGS. 3A-3C show association of IGF-1R levels with in vitro h10H5 response in colon cancer. In FIG. 3A, twenty seven colorectal cancer cells line were screened for in vitro sensitivity to h10H5 using an ATP based cell viability assay. The left axis and bar chart shows IGF-1R mRNA expression levels determined by microarray and the right axis and diamonds show the EC₅₀ for h10H5 in each cell line. FIG. 3B depicts percent inhibition of in vitro cell viability by h10H5 (x-axis) is correlated with IGF-1R mRNA levels determined by microarray (y-axis). Each point represents a single cell line. FIG. 3C shows pharmacodynamic response of sensitive HT-29 and insensitive HCT-116 cells to h10H5 treatment. Cells were treated with 1 mg/mL h10H5 for 24 hours and lysates used for immunoblotting with antibodies detecting the epitopes indicated to the right of the figure.

FIGS. 4A-4C show a gene expression signature of biomarkers of response to h10H5 in colorectal cancer cell lines. FIG. 4A is a heatmap showing expression of 60 genes identified through supervised analysis of gene expression data that distinguish h10H5 sensitive colorectal cells from resistant cells. Genes are shown on the y-axis and data was derived from log transformation and median centering for each gene. Red indicates high expression and green low expression according to z-scores. FIG. 4B shows the relationship of expression of a single candidate predictive biomarker, CD24, with growth inhibitory effects of h10H5 in cell lines. Bars indicate CD24 mRNA expression and diamonds the percent inhibition of cell viability observed in response to 1 mg/mL h10H5 treatment over three days. Error bars indicate standard deviations determined from four replicate experiments. FIG. 4C is a schematic of various classes of genes implicated in the h10H5 sensitivity and proposed relationship to signaling through the IGF-1R axis.

FIGS. 5A-5C show activity of h10H5 in colorectal xenograft and primary tumor explant models. FIG. 5A depicts Colo-205 tumors cells and CXF-280 primary colorectal tumor explant tissue were profiled on gene expression microarrays and data are shown for IGF-1R and the IGF-II. Colo-205 is a high receptor expression model and CXF-280 a high ligand expressing model. FIG. 5B shows 14 day daily dosing of flank xenografted Colo-205 high IGF-1R cells with h10H5 substantially reduced tumor growth in a dose-dependent manner. FIG. 5C shows a 14 day daily dosing of the human primary tumor explant xenograft model CXF-280 with h10H5 resulted in substantial reduction of tumor growth compared to animals dosed with vehicle or a control antibody.

FIGS. 6A-6D depict diagnostic assays for patient stratification in clinical trials. FIG. 6A reveals agreement between protein staining intensity with an IGF-1R IHC assay with mRNA levels in 42 breast cancer cell lines. Each point represents a cell line and IHC category (1+, 2+, 3+) is shown on the x-axis and IGF-1R mRNA levels on the y-axis. Examples of IHC (1+) and IHC (3+) staining are shown for the cell lines EVSA-T and BT474. FIG. 6B provides examples of low (1+), moderate (2+), and high (3+) IHC staining in neoplastic breast tissue samples. FIG. 6C show distribution of low, moderate and high IHC staining in a panel of breast and colorectal tumor samples. FIG. 6D shows qRT-PCR with a panel of biomarkers including IGF-1R, IGF-II, IRS1 and IRS2 was performed on a set of formalin fixed paraffin embedded colorectal tumors. The heatmap is color coded by z-scores as indicated in the figure.

FIG. 7 shows IGF-1 mediated growth stimulation index in breast cancer cell lines.

FIGS. 8A-8D depict dependence on IRS1 expression and signaling in h10H5 sensitive cell lines.

FIG. 9 reveals quantitation of downstream pathway modulation in response to h10H5.

FIG. 10 shows that components of the IGF-1R colorectal response signature are differentially expressed in MCF-7 cells treated with IGF-I.

FIG. 11 depicts expression of IGF-1R and IGF-II in xenograft models used to assess h10H5 anti-tumor activity.

FIG. 12 shows validation of qRT-PCR primer probe sets by comparing results from formalin fixed paraffin embedded (FFPE) cell lines with microarray chip data from fresh frozen cell line DNA.

### DETAILED DESCRIPTION OF THE PREFERRED EMBODIMENTS

### Definitions

“Insulin-like growth factor-I receptor” or “IGF-1R” is defined herein as a mammalian biologically active polypeptide, which, if human, has the amino acid sequence of SEQ ID NO:67 of U.S. Pat. No. 6,468,790. Preferably, the IGF-1R herein referred to is human.

“IGF” or “insulin-like growth factor” refers to IGF-I and IGF-II, which bind to IGF-1R and are well known in the literature, e.g., U.S. Pat. No. 6,331,609 and U.S. Pat. No. 6,331,414. They are normally mammalian as used herein, and most preferably human.

An “IGF-1R inhibitor” is a compound or composition which inhibits biological activity of IGF-1R. Preferably the inhibitor is an antibody or small molecule which binds IGF-1R. IGF-1R inhibitors can be used to modulate one or more aspects of IGF-1R-associated effects, including but not limited to IGF-1R activation, downstream molecular signaling, cell proliferation, cell migration, cell survival, cell morphogenesis, and angiogenesis. These effects can be modulated by any biologically relevant mechanism, including disruption of ligand (e.g., IGF-I and/IGF-II), binding to IGF-1R, or receptor phosphorylation, and/or receptor multimerization. Generally, such IGF-1R inhibitors will block binding of IGF-I and/or IGF-II to IGF-1R. The preferred IGF-1R inhibitor herein is an antibody, such as a human, humanized or chimeric antibody which binds IGF-1R. Examples of such antibodies include: human IgG1 antibody R1507 (Roche), human IgG2 antibody CP-751,871 (Pfizer), humanized antibody MK-0646 (Merck/Pierre Fabre), human IgG1 antibody IMC-A12 (Imclone), human antibody SCH717454 (Schering-Plough), human antibody AMG 479 (Amgen), fully human non-glycosylated IgG4.P antibody BIIB-022 (Biogen/IDEC), EM-164/AVE1642 (ImmunoGen/Sanofi), h7C10/F50035 (Merck/PierreFabre), humanized antibody AVE-1642 (Sanofi-Aventis), and humanized antibody 10H5 (Genentech). Examples of IGF-1R tyrosine kinase inhibitors include: reversible ATP-competitior INSM-18 (INSMED), oral small molecule XL-228 (Exelixis), oral small molecule, reversible ATP-competitor OSI-906 (QPIP) (OSI), A928605 (Abbott), GSK-665,602 and GSK-621,659 (Glaxo-Smith Kline), oral small molecule reversible ATP-competitors BMS-695,735, BMS-544,417, BMS-536,924, and BMS-743,816 (Bristol Myers Squibb), reversible ATP-competitors NOV-AEW-541, and NOV-ADW-742 (Novartis), antisense therapeutic ATL-1101 (Antisense Therapeutics), and HotSpot pharmaphore ANT-429 (Antyra).

“Blocking the interaction of an insulin-like growth factor (IGF) with IGF-1R” refers to interfering with the binding of an IGF to IGF-1R, whether complete or partial interfering or inhibiting.

A “biomarker” is a molecule produced by diseased cells, e.g. by cancer cells, whose expression is useful for identifying a patient who can benefit from therapy with a drug, such as an IGF1-R inhibitor. Positive expression of the biomarker, as well as increased (or decreased) level relative to cancer cells of the same cancer type can be used to identify patients for therapy. Biomarkers include intracellular molecules (e.g. ISR1 and ISR2), membrane bound molecules (e.g. IGF-1R) and soluble molecules (e.g. IGF-II). The present invention specifically contemplates combining one or more biomarkers to identify patients most likely to respond to IGF-1R therapy.

“Insulin receptor substrate adaptor 1” or “IRS1” is a transducer and/or amplifier of IGF-1R signaling, which recruits signaling complexes and results in proliferative and anti-apoptotic cellular responses. The IRS1 protein structure is disclosed in Sun et al. “Structure of the insulin receptor substrate IRS-1 defines a unique signal transduction protein.” Nature 352: 73-77 (1991): PubMed ID: 1648180.

“Insulin receptor substrate adaptor 2” or “IRS2” also transduces and/or amplifies IGF-1R signaling, recruits signaling complexes, and results in proliferative and anti-apoptotic cellular responses. The protein structure of IRS2 is disclosed in Sun et al. “Role of IRS-2 in insulin and cytokine signaling” Nature 377: 173-177 (1995); PubMed ID: 7675087

Protein “expression” refers to conversion of the information encoded in a gene into messenger RNA (mRNA) and then to the protein.

Herein, a sample or cell that “expresses” a protein of interest (such as a IGF-1R or the other biomarkers disclosed herein) is one in which mRNA encoding the protein, or the protein, including fragments thereof, is determined to be present in the sample or cell.

A sample, cell, tumor, or cancer which expresses a biomarker “at a level above the median” is one in which the level of biomarker expression is considered “high expression” to a skilled person for that type of cancer. In one embodiment, such level will be in the range from greater than 50% to about 100%, e.g. from about 75% to about 100% relative to biomarker level in a population of samples, cells, tumors, or cancers of the same cancer type. In one embodiment, e.g. for IRS1 and IRS2, such high expression will be at least one standard deviation above the median. According to the IHC assay in the example below, such “high expressing” tumor samples may express IGF-1R at a 2+ or 3+ level.

A sample, cell, tumor or cancer which expresses a biomarker such as IGF-1R “at a level below the median” for a type of cancer, such as breast cancer, is one in which the level of biomarker expression is considered “low expression” to a skilled person for that type of cancer. In one embodiment, such level will be in the range from less than 50% to about 0%, e.g. from about 25% to about 0% relative to biomarker level in a population of samples, cells, tumors, or cancers of the same cancer type. According to the IHC assay in the example below, such “low expressing” tumor samples may express IGF-1R at a 0 or 1+ level.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “microarray” refers to an ordered arrangement of hybridizable array elements, preferably polynucleotide probes, on a substrate.

An “effective response” and similar wording refers to a response to the IGF-1R inhibitor that is significantly higher than a response from a patient that does not express a certain biomarker at the designated level.

An “advanced” cancer is one which has spread outside the site or organ of origin, either by local invasion or metastasis.

A “refractory” cancer is one which progresses even though an anti-tumor agent, such as a chemotherapeutic agent, is being administered to the cancer patient.

A “recurrent” cancer is one which has regrown, either at the initial site or at a distant site, after a response to initial therapy.

Herein, a “patient” is a human patient. The patient may be a “cancer patient,” i.e. one who is suffering or at risk for suffering from one or more symptoms of cancer.

A “tumor sample” herein is a sample derived from, or comprising tumor cells from, a patient's tumor. Examples of tumor samples herein include, but are not limited to, tumor biopsies, circulating tumor cells (CTCs), plasma, serum, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

A “fixed” tumor sample is one which has been histologically preserved using a fixative.

A “formalin-fixed” tumor sample is one which has been preserved using formaldehyde as the fixative.

An “embedded” tumor sample is one surrounded by a firm and generally hard medium such as paraffin, wax, celloidin, or a resin. Embedding makes possible the cutting of thin sections for microscopic examination or for generation of tissue microarrays (TMAs).

A “paraffin-embedded” tumor sample is one surrounded by a purified mixture of solid hydrocarbons derived from petroleum.

Herein, a “frozen” tumor sample refers to a tumor sample which is, or has been, frozen.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol., 222: 581-597 (1992); Sidhu et al., J. Mol. Biol., 338(2): 299-310 (2004); Lee et al., J. Mol. Biol., 340(5): 1073-1093 (2004); Fellouse, Proc. Natl. Acad. Sci. USA, 101(34): 12467-12472 (2004); and Lee et al., J. Immunol. Methods, 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90: 2551 (1993); Jakobovits et al., Nature, 362: 255-258 (1993); Bruggemann et al., Year in Immunol., 7:33 (1993); U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnol., 14: 845-851 (1996); Neuberger, Nature Biotechnol., 14: 826 (1996); and Lonberg and Huszar, Intern. Rev. Immunol., 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., Proc. Natl. Acad. Sci. USA, 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region (HVR) of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature, 321:522-525 (1986); Riechmann et al., Nature, 332:323-329 (1988); and Presta, Curr. Op. Struct. Biol., 2:593-596 (1992). See also, for example, Vaswani and Hamilton, Ann. Allergy, Asthma & Immunol., 1:105-115 (1998); Harris, Biochem. Soc. Transactions, 23:1035-1038 (1995); Hurle and Gross, Curr. Op. Biotech., 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 227:381 (1991); Marks et al., J. Mol. Biol., 222:581 (1991)). Also available for the preparation of human monoclonal antibodies are methods described in Cole et al., Monoclonal Antibodies and Cancer Therapy, Alan R. Liss, p. 77 (1985) and Boerner et al., J. Immunol., 147(1):86-95 (1991). See also van Dijk and van de Winkel, Curr. Opin. Pharmacol., 5: 368-374 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

An “affinity-matured” antibody is an antibody with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 10:779-783 (1992) describes affinity maturation by VH- and VL-domain shuffling. Random mutagenesis of HVR and/or framework residues is described by, for example: Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

A “native-sequence Fc region” comprises an amino acid sequence identical to the amino acid sequence of an Fc region found in nature. Native-sequence human Fc regions include a native-sequence human IgG1 Fc region (non-A and A allotypes), native-sequence human IgG2 Fc region, native-sequence human IgG3 Fc region, and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and more preferably at least about 90% homology therewith, and most preferably at least about 95% homology therewith.

The terms “cancer” and “cancerous” refer to or describe the physiological condition in humans that is typically characterized by unregulated cell growth.

A “cancer type” herein refers to a particular category or indication of cancer. Examples of such cancer types include, but are not limited to prostate cancer such as hormone-resistant prostate cancer, osteosarcoma, breast cancer, endometrial cancer, lung cancer such as non-small cell lung carcinoma, ovarian cancer, colorectal cancer, pediatric cancer, pancreatic cancer, bone cancer, bone or soft tissue sarcoma or myeloma, bladder cancer, primary peritoneal carcinoma, fallopian tube carcinoma, Wilm's cancer, benign prostatic hyperplasia, cervical cancer, squamous cell carcinoma, head and neck cancer, synovial sarcoma, liquid tumors, multiple myeloma, cervical cancer, kidney cancer, liver cancer, synovial carcinoma, and pancreatic cancer. Liquid tumors herein include acute lymphocytic leukemia (ALL) or chronic milogenic leukemia (CML); liver cancers herein include hepatoma, hepatocellular carcinoma, cholangiocarcinoma, angiosarcoma, hemangiosarcoma, or hepatoblastoma. Other cancers to be treated include multiple myeloma, ovarian cancer, osteosarcoma, cervical cancer, prostate cancer, lung cancer, kidney cancer, liver cancer, synovial carcinoma, and pancreatic cancer. Cancers of particular interest herein are breast cancer and colorectal cancer.

“Colorectal cancer” includes colon cancer, rectal cancer, and colorectal cancer (i.e. cancer of both the colon and rectal areas).

The terms “therapeutically effective amount” or “effective amount” refer to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival, result in an objective response (including a partial response, PR, or complete respose, CR), improve survival (including overall survival and progression free survival) and/or improve one or more symptoms of cancer. Most preferably, the therapeutically effective amount of the drug is effective to improve progression free survival (PFS) and/or overall survival (OS).

“Survival” refers to the patient remaining alive, and includes overall survival as well as progression free survival.

“Overall survival” refers to the patient remaining alive for a defined period of time, such as 1 year, 5 years, etc from the time of diagnosis or treatment.

“Progression free survival” refers to the patient remaining alive, without the cancer progressing or getting worse.

By “extending survival” is meant increasing overall or progression free survival in a treated patient relative to an untreated patient (i.e. relative to a patient not treated with IGF-1R inhibitor), or relative to a patient who does not express biomarker(s) at the designated level, and/or relative to a patient treated with an approved anti-tumor agent used to treat the particular cancer of interest.

An “objective response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

The term “cytotoxic agent” as used herein refers to a substance that inhibits or prevents the function of cells and/or causes destruction of cells. The term includes radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), and toxins such as small-molecule toxins or enzymatically active toxins of bacterial, fungal, plant, or animal origin, or fragments thereof.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); dynemicin, including dynemicin A; an esperamicin; as well as neocarzinostatin chromophore and related chromoprotein enediyne antibiotic chromophores), other antibiotics such as aclacinomycin, actinomycin, authramycin, azaserine, bleomycin, cactinomycin, carabicin, caminomycin, carzinophilin, chromomycin, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), and deoxydoxorubicin), epirubicin, esorubicin, idarubicin, marcellomycin, mitomycins such as mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

An “estrogen inhibitor” is a molecule or composition which inhibits estrogen or estrogen receptor biological function. Generally, such inhibitors will bind to either estrogen or the estrogen receptor (ER receptor), but agents which have an indirect affect on estrogen receptor function, including the aromatase inhibitors and estrogen receptor down-regulators are included in this class of drugs. Examples of estrogen inhibitors herein include: selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); estrogen receptor down-regulators (ERDs); estrogen receptor antagonists such as fulvestrant (FASLODEX®); aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). The preferred estrogen inhibitors herein are estrogen and fulvestrant.

A “growth-inhibitory agent” refers to a compound or composition that inhibits growth of a cell, which growth depends on receptor activation either in vitro or in vivo. Thus, the growth-inhibitory agent includes one that significantly reduces the percentage of receptor-dependent cells in S phase. Examples of growth-inhibitory agents include agents that block cell-cycle progression (at a place other than S phase), such as agents that induce G1 arrest and M-phase arrest. Classical M-phase blockers include the vincas and vinca alkaloids (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA-alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anti-cancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb).

The term “cytokine” is a generic term for proteins released by one cell population that act on another cell as intercellular mediators. Examples of such cytokines are lymphokines, monokines, interleukins (ILs) such as IL-1, IL-1α, IL-2, IL-3, IL-4, IL-5, IL-6, IL-7, IL-8, IL-9, IL-11, IL-12, and IL-15, including PROLEUKIN® rIL-2, a tumor-necrosis factor such as TNF-α or TNF-β, and other polypeptide factors including leukocyte-inhibitory factor (LIF) and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

A “package insert” refers to instructions customarily included in commercial packages of medicaments that contain information about the indications, usage, dosage, administration, contraindications, other medicaments to be combined with the packaged product, and/or warnings concerning the use of such medicaments, etc.

### MODES FOR CARRYING OUT THE INVENTION

### Invention Aspects

### Biomarkers and Diagnostic Methods

Various aspects of the biomarker selection methods of this invention supported by the experimental data herein include the identification of patients who can benefit from therapy with an IGF-1R inhibitor (particularly an IGF-1R antibody) as follows:

(a) identifying a patient with cancer (e.g. breast or colorectal cancer) for therapy, where the patient's cancer has been shown to express, at a level above the median for the type of cancer being treated, two or more biomarkers selected from the group consisting of IGF-1R, IGF-II, TRS1 and IRS2;
(b) identifying a patient with breast cancer for therapy, provided the patient's cancer has not been found to express IGF-1R at a level below the median for breast cancer;
(c) identifying a patient with breast cancer for therapy, where the patient has been shown to express one or more biomarkers selected from the group consisting of IGF-1R, IRS1, IRS2, IGF-II, and estrogen receptor, at level above the median for breast cancer;
(d) identifying a patient with colorectal cancer for therapy, where patient's cancer expresses IGF-1R at a level greater than the median level for IGF-1R expression in colorectal cancer;
(e) identifying a patient with colorectal cancer for therapy, where the patient's cancer expresses one to eleven (e.g. two or more, three or more, four or more, five or more, six or more, seven or more eight or more, nine or more, ten, or eleven) biomarkers selected from the group consisting of: TOB1, CD24, MAP2K6, SMAD6, TNFSF10, PMP22, CTSL1, ZMYM2, PALM2, ICAM1, and GBE1. Optionally, the patient has also been shown to express IGF-1R at a level above the median for colorectal cancer.

According to specific embodiments of the invention herein, the patient's cancer expresses IRS1 and/or IRS2 at least one standard deviation above the median. In one embodiment, the patient's cancer expresses IGF-1R, and either or both of IRS1 or IRS2, above the median. In another embodiment, the patient's cancer expresses IGF-II, and either or both of IRS1 or IRS2, above the median.

Preferably the cancer is breast or colorectal cancer.

Biomarker expression is preferably determined using immunohistochemistry (IHC), or polymerase chain reaction (PCR), preferably quantitative real time polymerase chain reaction (qRT-PCR).

The methods herein involve obtaining a biological sample from the patient and testing it for biomarker expression, such sample may be from a patient biopsy, or circulating tumor cells (CTLs), serum, or plasma from the patient.

The median or percentile expression level can be determined essentially contemporaneously with measuring biomarker expression, or may have been determined previously.

Prior to to therapeutic methods described below, biomarker expression level(s) in the patient's cancer is/are assessed. Generally, a biological sample is obtained from the patient in need of therapy, which sample is subjected to one or more diagnostic assay(s), usually at least one in vitro diagnostic (IVD) assay. However, other forms of evaluating biomarker expression, such as in vivo diagnosis, are expressly contemplated herein. The biological sample is usually a tumor sample, preferably from a breast or colorectal cancer patient.

The biological sample herein may be a fixed sample, e.g. a formalin fixed, paraffin-embedded (FFPE) sample, or a frozen sample.

Various methods for determining expression of mRNA or protein include, but are not limited to: immunohistochemistry (IHC), gene expression profiling, polymerase chain reaction (PCR) including quantitative real time PCR (qRT-PCR), microarray analysis, serial analysis of gene expression (SAGE), MassARRAY, Gene Expression Analysis by Massively Parallel Signature Sequencing (MPSS), proteomics, etc. Preferably protein or mRNA is quantified. mRNA analysis is preferably performed using the technique of polymerase chain reaction (PCR), or by microarray analysis. Where PCR is employed, a preferred form of PCR is quantitative real time PCR (qRT-PCR).

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR. Finally, the data are analyzed to identify the best treatment option(s) available to the patient on the basis of the characteristic gene expression pattern identified in the tumor sample examined.

Various exemplary methods for determining gene expression will now be described in more detail.

### Immunohistochemistry

Immunohistochemistry (IHC) methods are suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available. The Example below provides an IHC assay for IGF-1R protein.

### Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker &Barnes, Methods in Molecular Biology 106:247-283 (1999)); RNAse protection assays (Hod, Biotechniques 13:852-854 (1992)); and polymerase chain reaction (PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

### Polymerase Chain Reaction (PCR)

Of the techniques listed above, a sensitive and flexible quantitative method is PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif., USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® PCR can be performed using commercially available equipment, such as, for example, ABI PRISM 7700® Sequence Detection System® (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700® Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and P-actin.

A more recent variation of the PCR technique is quantitative real time PCR (qRT-PCR), which measures PCR product accumulation through a dual-labeled fluorigenic probe (i.e., TAQMAN® probe). Real time PCR is compatible both with quantitative competitive PCR, where internal competitor for each target sequence is used for normalization, and with quantitative comparative PCR using a normalization gene contained within the sample, or a housekeeping gene for PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: Godfrey et al., J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W., Genome Res. 12(4):656-64 (2002), or by the BLAST software including its variations. Subsequent steps follow well established methods of PCR primer and probe design.

### Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GENCHIP™ technology, or Incyte's microarray technology.

The development of microarray methods for large-scale analysis of gene expression makes it possible to search systematically for molecular markers of cancer classification and outcome prediction in a variety of tumor types.

### Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

### MassARRAY Technology

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dispensed on a chip array that is pre-loaded with the components needed for MALTI-TOF MS sample preparation. The various cDNAs present in the reaction are quantitated by analyzing the peak areas in the mass spectrum obtained.

### Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 microgram diameter microbeads. First, a microbead library of DNA templates is constructed by in vitro cloning. This is followed by the assembly of a planar array of the template-containing microbeads in a flow cell at a high density (typically greater than 3×106 microbeads/cm2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

### Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

### In Vivo Assays

Biomarker expression may also be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

### IGF-1R Inhibitors

As noted above in the background section, many different IGF-1R inhibitors are known in the art. According to the preferred embodiment of the invention, preferably the IGF-1R inhibitor is an antibody which binds to IGF-1R.

Preferred antibodies bind IGF-1R with an affinity of at least about 10⁻¹² M, more preferably at least about 10⁻¹³ M. The antibodies also preferably are of the IgG isotype, such as IgG1, IgG2a, IgG2b, or IgG3, more preferably human IgG, and most preferably IgG1 or IgG2a (most preferably human IgG1 or IgG2a).

The antibodies herein are preferably chimeric, human, or humanized. The antibodies of interest include intact antibodies as well as antibody fragments that bind IGF-1R. Such antibodies including fragments may be naked or conjugated with one or more heterologous molecules, e.g. with one or more cytotoxic agent(s) as in an antibody drug conjugate (ADC).

### Fc Variant Antibodies

The antibodies of the present invention may have a native-sequence Fc region. However, they may further comprise other amino acid substitutions that, e.g., improve or reduce other Fc function or further improve the same Fc function, increase antigen-binding affinity, increase stability, alter glycosylation, or include allotypic variants. The antibodies may further comprise one or more amino acid substitutions in the Fc region that result in the antibody exhibiting one or more of the properties selected from increased FcγR binding, increased ADCC, increased CDC, decreased CDC, increased ADCC and CDC function, increased ADCC but decreased CDC function (e.g., to minimize infusion reaction), increased FcRn binding, and increased serum half life, as compared to the polypeptide and antibodies that have wild-type Fc. These activities can be measured by the methods described herein.

For additional amino acid alterations that improve Fc function, see, e.g., U.S. Pat. No. 6,737,056. Any of the antibodies of the present invention may further comprise at least one amino acid substitution in the Fc region that decreases CDC activity, for example, comprising at least the substitution K322A (see, e.g., U.S. Pat. No. 6,528,624). Mutations that improve ADCC and CDC include S298A/E333A/K334A also referred to herein as the triple Ala mutant. K334L increases binding to CD 16. K322A results in reduced CDC activity. K326A or K326W enhances CDC activity. D265A results in reduced ADCC activity. Glycosylation variants that increase ADCC function are described, e.g., in WO 2003/035835. Stability variants are variants that show improved stability with respect to e.g., oxidation and deamidation. See also WO 2006/105338 for additional Fc variants.

### Glycosylation Variants

A further type of amino acid variant of the antibody alters the original glycosylation pattern of the antibody. Such altering includes deleting one or more carbohydrate moieties found in the antibody, and/or adding one or more glycosylation sites that are not present in the antibody. Glycosylation variants that increase ADCC function are described, e.g., in WO 2003/035835. See also US 2006/0067930.

Where the antibody comprises an Fc region, the carbohydrate attached thereto may be altered. For example, antibodies with a mature carbohydrate structure that lacks fucose attached to an Fc region of the antibody are described in US 2003/0157108 (Presta). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in, e.g., WO 2003/011878, Jean-Mairet et al. and U.S. Pat. No. 6,602,684 (Umana et al.). Antibodies with at least one galactose residue in the oligosaccharide attached to an Fc region of the antibody are reported, for example, in WO 1997/30087 (Patel et al.). See, also, WO 1998/58964 (Raju) and WO 1999/22764 (Raju) concerning antibodies with altered carbohydrate attached to the Fc region thereof. See also US 2005/0123546 (Umana et al.); US 2004/0072290 (Umana et al.); US 2003/0175884 (Umana et al.); WO 2005/044859 (Umana et al.); and US 2007/0111281 (Sondermann et al.) on antigen-binding molecules with modified glycosylation, including antibodies with an Fc region containing N-linked oligosaccharides; and US 2007/0010009 (Kanda et al.)

One preferred glycosylation antibody variant herein comprises an Fc region wherein a carbohydrate structure attached to the Fc region has reduced fucose or lacks fucose, which may improve ADCC function. Specifically, antibodies are contemplated herein that have reduced fusose relative to the amount of fucose on the same antibody produced in a wild-type CHO cell. That is, they are characterized by having a lower amount of fucose than they would otherwise have if produced by native CHO cells. Preferably the antibody is one wherein less than about 10% of the N-linked glycans thereon comprise fucose, more preferably wherein less than about 5% of the N-linked glycans thereon comprise fucose, and most preferably, wherein none of the N-linked glycans thereon comprise fucose, i.e., wherein the antibody is completely without fucose, or has no fucose.

Such “defucosylated” or “fucose-deficient” antibodies may be produced, for example, by culturing the antibodies in a cell line such as that disclosed in, for example, US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; US 2006/0063254; US 2006/0064781; US 2006/0078990; US 2006/0078991; Okazaki et al. J. Mol. Biol. 336:1239-1249 (2004); and Yamane-Ohnuki et al., Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US 2003/0157108 A1 (Presta) and WO 2004/056312 A1 (Adams et al., especially at Example 11) and knockout cell lines, such as alpha-1,6-fucosyltransferase gene, FUT8-knockout CHO cells (Yamane-Ohnuki et al., Biotech. Bioeng. 87: 614 (2004)). See also Kanda et al., Biotechnol. Bioeng., 94: 680-8 (2006). US 2007/0048300 (Biogen-IDEC) discloses a method of producing aglycosylated Fc-containing polypeptides, such as antibodies, having desired effector function, as well as aglycosylated antibodies produced according to the method and methods of using such antibodies as therapeutics, all being applicable herein. Additionally, U.S. Pat. No. 7,262,039 relates to a polypeptide having an alpha-1,3-fucosyltransferase activity, including a method for producing a fucose-containing sugar chain using the polypeptide.

### Immunoconjugates

The invention also pertains to immunoconjugates, or antibody-drug conjugates (ADC), comprising an antibody conjugated to a cytotoxic agent such as a chemotherapeutic agent, a drug, a growth-inhibitory agent, a toxin (e.g., an enzymatically active toxin of bacterial, fungal, plant, or animal origin, or fragments thereof), or a radioactive isotope (i.e., a radioconjugate). Such ADC must show a safety profile that is acceptable.

The use of ADCs for the local delivery of cytotoxic or cytostatic agents, e.g., drugs to kill or inhibit tumor cells in the treatment of cancer (Syrigos and Epenetos Anticancer Research, 19:605-614 (1999); Niculescu-Duvaz and Springer, Adv. Drug Del. Rev., 26:151-172 (1997); U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., Lancet, 603-605 (1986); Thorpe, “Antibody Carriers Of Cytotoxic Agents In Cancer Therapy: A Review,” in Monoclonal Antibodies '84: Biological And Clinical Applications, A. Pinchera et al. (eds), pp. 475-506 (1985)). Maximal efficacy with minimal toxicity is sought thereby. Both polyclonal antibodies and monoclonal antibodies have been reported as useful in these strategies (Rowland et al., Cancer Immunol. Immunother., 21:183-187 (1986)). Drugs used in these methods include daunomycin, doxorubicin, methotrexate, and vindesine. Toxins used in antibody-toxin conjugates include bacterial toxins such as diphtheria toxin, plant toxins such as ricin, small molecule toxins such as geldanamycin (Mandler et al., J. Nat. Cancer Inst., 92(19):1573-1581 (2000); Mandler et al., Bioorganic & Med. Chem. Letters, 10:1025-1028 (2000); and Mandler et al., Bioconjugate Chem., 13: 786-791 (2002)), maytansinoids (EP 1391213 and Liu et al., Proc. Natl. Acad. Sci. USA, 93: 8618-8623 (1996)), and calicheamicin (Lode et al., Cancer Res., 58:2928 (1998) and Hinman et al., Cancer Res. 53:3336-3342 (1993)). Without being limited to any one theory, the toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, for example, WO 1994/11026.

Conjugates of an antibody and at least one small-molecule toxin, e.g., a calicheamicin, maytansinoid, trichothecene, or CC 1065, or derivatives of these toxins with toxin activity, are also included.

The ADCs herein are optionally prepared with cross-linker reagents such as, for example, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), which are commercially available (e.g., Pierce Biotechnology, Inc., Rockford, Ill.).

### Other Antibody Derivatives

The antibodies of the present invention can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Pharmaceutical Formulations

Therapeutic formulations of the antibodies herein are prepared for storage by mixing an antibody having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as, e.g., TWEEN™, PLURONICS™, or polyethylene glycol (PEG).

A further formulation and delivery method herein involves that described, for example, in WO 2004/078140, including the ENHANZE™ drug delivery technology (Halozyme Inc.). This technology is based on a recombinant human hyaluronidase (rHuPH20). rHuPH20 is a recombinant form of the naturally occurring human enzyme approved by the FDA that temporarily clears space in the matrix of tissues such as skin. That is, the enzyme has the ability to break down hyaluronic acid (HA), the space-filling “gel”-like substance that is a major component of tissues throughout the body. This clearing activity is expected to allow rHuPH20 to improve drug delivery by enhancing the entry of therapeutic molecules through the subcutaneous space. Hence, when combined or co-formulated with certain injectable drugs, this technology can act as a “molecular machete” to facilitate the penetration and dispersion of these drugs by temporarily opening flow channels under the skin. Molecules as large as 200 nanometers may pass freely through the perforated extracellular matrix, which recovers its normal density within approximately 24 hours, leading to a drug delivery platform that does not permanently alter the architecture of the skin.

Hence, the present invention includes a method of delivering an antibody herein to a tissue containing excess amounts of glycosaminoglycan, comprising administering a hyaluronidase glycoprotein (sHASEGP) (this protein comprising a neutral active soluble hyaluronidase polypeptide and at least one N-linked sugar moiety, wherein the N-linked sugar moiety is covalently attached to an asparagine residue of the polypeptide) to the tissue in an amount sufficient to degrade glycosaminoglycans sufficiently to open channels less than about 500 nanometers in diameter; and administering the antibody to the tissue comprising the degraded glycosaminoglycans.

In another embodiment, the invention includes a method for increasing the diffusion of an antibody herein that is administered to a subject comprising administering to the subject a sHASEGP polypeptide in an amount sufficient to open or to form channels smaller than the diameter of the antibody and administering the antibody, whereby the diffusion of the therapeutic substance is increased. The sHASEGP and antibody may be administered separately or simultaneously in one formulation, and consecutively in either order or at the same time.

Exemplary anti-IGF-1R antibody formulations may be made generally as set forth in WO 1998/56418, which include a liquid multidose formulation comprising an antibody at 40 mg/mL, 25 mM acetate, 150 mM trehalose, 0.9% benzyl alcohol, 0.02% polysorbate 20 surfactant at pH 5.0 that has a minimum shelf life of two years storage at 2-8° C. Another suitable anti-IGF-1R formulation comprises 10 mg/mL antibody in 9.0 mg/mL sodium chloride, 7.35 mg/mL sodium citrate dihydrate, 0.7 mg/mL polysorbate 80 surfactant, and Sterile Water for Injection, pH 6.5.

The antibody herein may also be formulated, for example, as described in WO 1997/04801, which teaches a stable lyophilized protein formulation that can be reconstituted with a suitable diluent to generate a high-protein concentration reconstituted formulation suitable for subcutaneous administration. Preferably, however, the antibody herein is formulated as described in U.S. Pat. No. 6,171,586. This patent teaches a stable aqueous pharmaceutical formulation comprising a therapeutically effective amount of an antibody not subjected to prior lyophilization, an acetate buffer from about pH 4.8 to about 5.5, a surfactant, and a polyol, wherein the formulation lacks a tonicifying amount of sodium chloride. The polyol is preferably a nonreducing sugar, more preferably trehalose or sucrose, most preferably trehalose, preferably at an amount of about 2-10% w/v. The antibody concentration in the formulation is preferably from about 0.1 to about 50 mg/mL, and the surfactant is preferably a polysorbate surfactant, preferably an amount of about 0.01-0.1% v/v. The acetate is preferably present in an amount of about 5-30 mM, more preferably about 10-30 mM. The formulation optionally further contains a preservative, which is preferably benzyl alcohol.

One especially preferred formulation herein is about 20 to 50 mg/mL antibody, sodium acetate in an amount of about 10-30 mM, pH about 4.8 to about 5.5, trehalose, and a polysorbate surfactant. One particularly preferred formulation herein is one in which the bulk concentration of the antibody is about 20 mg/mL and the formulation also contains about 20 mM sodium acetate, pH 5.3±0.3, about 200-300 mM trehalose, more preferably about 240 mM trehalose, and about 0.02% polysorbate 20 surfactant.

Lyophilized formulations adapted for subcutaneous administration are described in U.S. Pat. No. 6,267,958. Such lyophilized formulations may be reconstituted with a suitable diluent to a high protein concentration and the reconstituted formulation may be administered subcutaneously to the subject to be treated herein.

Crystallized forms of the antibody are also contemplated. See, for example, US 2002/0136719.

The formulation herein may also contain more than one active compound (a second medicament as noted herein) as necessary for the particular indication being treated, preferably those with complementary activities that do not adversely affect each other. For example, it may be desirable to further provide a cytotoxic agent, chemotherapeutic agent, cytokine antagonist, integrin antagonist, or immunosuppressive agent. The type and effective amounts of such second medicaments depend, for example, on the amount of antibody present in the formulation, the type of disease or disorder or treatment, the clinical parameters of the subjects, and other factors discussed above. These are generally used in the same dosages and with administration routes as described herein or about from about 1 to 99% of the heretofore employed dosages.

The active ingredients may also be entrapped in microcapsules prepared, e.g., by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nano-capsules) or in macroemulsions. Such techniques are disclosed, for example, in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in-vivo administration must be sterile. This is readily accomplished by filtration through sterile filtration membranes.

### Uses

The antibody may be a naked antibody or alternatively is conjugated with another molecule, e.g. a cytotoxic agent if the resulting immunoconjugate has an acceptable safety profile. In certain aspects, the immunoconjugate and/or antigen to which it is bound is/are internalized by the cell, resulting in increased therapeutic efficacy of the immunoconjugate in killing the target cell to which it binds. In one aspect, the cytotoxic agent targets or interferes with nucleic acid in the target cell. Examples of such cytotoxic agents include any chemotherapeutic agents noted herein (e.g., a maytansinoid or a calicheamicin), a radioactive isotope, a ribonuclease, or a DNA endonuclease. Preferably, the antibodies herein are conjugated to a cell toxin and/or a radioelement.

In one embodiment, the subject has never been previously administered any drug(s), such as immunosuppressive agent(s), to treat the disorder. In a still further aspect, the subject or patient is not responsive to therapy for the disorder. In another embodiment, the subject or patient is responsive to therapy for the disorder.

In another embodiment, the subject or patient has been previously administered one or more drug(s) to treat the disorder. In a further embodiment, the subject or patient was not responsive to one or more of the medicaments that had been previously administered. Such drugs to which the subject may be non-responsive include, for example, chemotherapeutic agents, cytotoxic agents, anti-angiogenic agents, immunosuppressive agents, pro-drugs, cytokines, cytokine antagonists, cytotoxic radiotherapies, corticosteroids, anti-emetics, cancer vaccines, analgesics, anti-vascular agents, growth-inhibitory agents, epidermal growth factor receptor (EGFR) inhibitors such as erlotinib, an Apo2L/TRAIL DR5 agonist (such as apomab, a DR-5-targeted dual proapoptotic receptor agonist), or antagonists to IGF-1R (e.g., a molecule that inhibits or reduces a biological activity of IGF-1R, such as one that substantially or completely inhibits, blocks, or neutralizes one or more biological activities of IGF-1R). More particularly, the drugs to which the subject may be non-responsive include chemotherapeutic agents, cytotoxic agents, anti-angiogenic agents, immunosuppressive agents, EGFR inhibitors such as erlotinib, apomab, or antagonists to IGF-1R. Preferably, such IGF-1R antagonists do not include an antibody of this invention (such IGF-1R antagonists include, for example, small-molecule inhibitors of IGF-1R, or anti-sense oligonucleotides, antagonistic peptides, or antibodies to IGF-1R that are not the antibodies of this invention, as noted, for example, in the background section above). In a further aspect, such IGF-1R antagonists include an antibody of this invention, such that re-treatment is contemplated with one or more antibodies of this invention.

In yet another embodiment, the antibody herein is the only medicament administered to the subject to treat the disorder. In a further aspect, the antibody herein is one of the medicaments used to treat the disorder. Preferably, the subject being treated herein is human.

The antibodies herein are especially useful in treating cancer and inhibiting tumor growth. Examples of types of cancers treatable herein are provided hereinabove, including preferred cancers, such as particularly breast or colorectal cancers.

### Dosage

For the prevention or treatment of disease, the appropriate dosage of the IGF-1R inhibitor of the invention (when used alone or in combination with a second medicament as noted below) will depend, for example, on the type of cancer to be treated, the type of antibody, the severity and course of the cancer, whether the antibody is administered for preventive or therapeutic purposes, previous therapy, the patient's clinical history and response to the antibody, and the discretion of the attending physician. The dosage is preferably efficacious for the treatment of that indication while minimizing toxicity and side effects.

The inhibitor is suitably administered to the patient at one time or over a series of treatments. Depending on the type and severity of the disease, about 1 μg/kg to 500 mg/kg (preferably about 0.1 mg/kg to 400 mg/kg) of an IGF-1R antibody is an initial candidate dosage for administration to the patient, whether, for example, by one or more separate administrations, or by continuous infusion. One typical daily dosage might range from about 1 μg/kg to 500 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 400 mg/kg. Thus, one or more doses of about 0.5 mg/kg, 2.0 mg/kg, 4.0 mg/kg or 10 mg/kg or 50 mg/kg or 100 mg/kg or 300 mg/kg or 400 mg/kg (or any combination thereof) may be administered to the patient. Such doses may be administered intermittently, e.g., every week or every three weeks (e.g., such that the patient receives from about two to about twenty, e.g., about six doses of the antibody). An initial higher loading dose, followed by one or more lower doses, may be administered. An exemplary dosing regimen comprises administering an initial loading dose of about 4 to 500 mg/kg, followed by a weekly maintenance dose of about 2 to 400 mg/kg of the antibody. However, other dosage regimens may be useful. The progress of this therapy is easily monitored by conventional techniques and assays.

For the treatment of cancer, the therapeutically effective dosage will typically be in the range of about 50 mg/m² to about 3000 mg/m², preferably about 50 to 1500 mg/m², more preferably about 50-1000 mg/m². In one embodiment, the dosage range is about 125-700 mg/m². In different embodiments, the dosage is about any one of 50 mg/dose, 80 mg/dose, 100 mg/dose, 125 mg/dose, 150 mg/dose, 200 mg/dose, 250 mg/dose, 275 mg/dose, 300 mg/dose, 325 mg/dose, 350 mg/dose, 375 mg/dose, 400 mg/dose, 425 mg/dose, 450 mg/dose, 475 mg/dose, 500 mg/dose, 525 mg/dose, 550 mg/dose, 575 mg/dose, or 600 mg/dose, or 700 mg/dose, or 800 mg/dose, or 900 mg/dose, or 1000 mg/dose, or 1500 mg/dose.

In treating disease, IGF-1R antibodies of the invention can be administered to the patient chronically or intermittently, as determined by the physician of skill in the disease.

The antibodies herein may be administered at a frequency that is within the skill and judgment of the practicing physician, depending on various factors noted above, for example, the dosing amount. This frequency includes twice a week, three times a week, once a week, bi-weekly, or once a month, In a preferred aspect of this method, the antibody is administered no more than about once every other week, more preferably about once a month.

### Route of Administration

The antibodies used in the methods of the invention (as well as any second medicaments) are administered to a subject or patient, including a human patient, in accord with suitable methods, such as those known to medical practitioners, depending on many factors, including whether the dosing is acute or chronic. These routes include, for example, parenteral, intravenous administration, e.g., as a bolus or by continuous infusion over a period of time, by subcutaneous, intramuscular, intra-arterial, intraperitoneal, intrapulmonary, intracerebrospinal, intra-articular, intrasynovial, intrathecal, intralesional, or inhalation routes (e.g., intranasal). Parenteral infusions include intramuscular, intravenous, intraarterial, intraperitoneal, or subcutaneous administration. In addition, the antibody is suitably administered by pulse infusion, particularly with declining doses of the antibody. Preferred routes herein are intravenous or subcutaneous administration.

More preferably, the antibody is administered intravenously, still more preferably about every 21 days, still more preferably over about 30 to 90 minutes. In another embodiment, such iv-infused or treated subjects have cancer, preferably advanced or metastatic solid tumors, more preferably breast or colorectal cancer. Additionally, such treated subjects preferably have progressed on prior therapy (such as, for example, chemotherapy) and/or preferably have not been previously treated with EGFR inhibitors such as erlotinib or apomab, or are those for whom there is no effective therapy.

In one embodiment, the antibody herein is administered by intravenous infusion, and more preferably with about 0.9 to 20% sodium chloride solution as an infusion vehicle.

### Combination Therapy

In any of the methods herein, one may administer to the subject or patient along with the antibody herein an effective amount of a second medicament (where the antibody herein is a first medicament), which is another active agent that can treat the condition in the subject that requires treatment. For instance, an antibody of the invention may be co-administered with another antibody, chemotherapeutic agent(s) (including cocktails of chemotherapeutic agents), cytotoxic agent(s), anti-angiogenic agent(s), cytokine(s), cytokine antagonist(s), and/or growth-inhibitory agent(s). The type of such second medicament depends on various factors, including the type of cancer, the severity of the disease, the condition and age of the patient, the type and dose of first medicament employed, etc.

According to a preferred embodiment of combination therapy, the invention concerns treating breast cancer in a human patient by administering a combination of an IGF-1R inhibitor and an estrogen inhibitor (such as tamoxifen and fulvestrant), wherein the combination results in a synergistic effect in the patient. The data below supports such synergy of this combination.

The IGF-1R inhibitor may be combined with an anti-VEGF antibody (e.g., AVASTIN®), an Apo2L/TRAIL DR5 agonist (such as apomab, a DR-5-targeted dual proapoptotic receptor agonist), and/or anti-ErbB antibodies (e.g. HERCEPTIN® trastuzumab anti-HER2 antibody or an anti-HER2 antibody that binds to Domain II of HER2, such as pertuzumab anti-HER2 antibody or erlotinib (TARCEVA™)) in a treatment scheme, e.g., in treating breast or colorectal cancer. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies.

Treatment with a combination of the antibody herein with one or more second medicaments preferably results in an improvement in the signs or symptoms of cancer. For instance, such therapy may result in an improvement in survival (overall survival and/or progression-free survival) relative to a patient treated with the second medicament only (e.g., a chemotherapeutic agent only), and/or may result in an objective response (partial or complete, preferably complete). Moreover, treatment with the combination of an antibody herein and one or more second medicament(s) preferably results in an additive, and more preferably synergistic (or greater than additive), therapeutic benefit to the patient. Preferably, in this combination method the timing between at least one administration of the second medicament and at least one administration of the antibody herein is about one month or less, more preferably, about two weeks or less.

For treatment of cancers, the second medicament is preferably another antibody, chemotherapeutic agent (including cocktails of chemotherapeutic agents), cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, and/or growth-inhibitory agent. The cytotoxic agent includes a small-molecule inhibitor to IGF-1R as well as other peptides and anti-sense oligonucleotides and other molecules used to target IGF-1R, such as, e.g., BMS-536924, BMS-55447, BMS-636924, AG-1024, OSIP Compound 2/0S1005, NVP-ADW-742 or NVP-AEW541 (see AACR annual meeting abstracts, Apr. 1-6, 2006), bicyclo-pyrazole inhibitors such as those described in WO 2007/099171, pyrazolo-pyridine derivative inhibitors such as those described in WO 2007/099166, or another IGF-1R antibody that those claimed herein, such as those set forth above, an agent interacting with DNA, the anti-metabolites, the topoisomerase I or II inhibitors, a hyaluronidase glycoprotein as an active delivery vehicle as set forth in, for example, WO 2004/078140, or the spindle inhibitor or stabilizer agents (e.g., preferably vinca alkaloid, more preferably selected from vinblastine, deoxyvinblastine, vincristine, vindesine, vinorelbine, vinepidine, vinfosiltine, vinzolidine and vinfunine), or any agent used in chemotherapy such as 5-FU, a taxane, doxorubicin, or dexamethasone.

In another embodiment, the second medicament is another antibody used to treat cancer such as those directed against the extracellular domain of the HER2/neu receptor, e.g., trastuzumab, or one of its functional fragments, pan-HER inhibitor, a Src inhibitor, a MEK inhibitor, or an EGFR inhibitor (e.g., an anti-EGFR antibody (such as one inhibiting the tyrosine kinase activity of the EGFR), which is preferably the mouse monoclonal antibody 225, its mouse-man chimeric derivative C225, or a humanized antibody derived from this antibody 225 or derived natural agents, dianilinophthalimides, pyrazolo- or pyrrolopyridopyrimidines, quinazilines, gefitinib (IRESSA®), Apo2 ligand or tumor necrosis factor-related apoptosis-inducing ligand (Apo2L/TRAIL), a dual pro-apoptotic receptor agonist designed to activate both pro-apoptotic receptors DR4 and DR5 (including the polypeptides disclosed in WO 1997/01633, WO 1997/25428, and WO 2001/00832, where Apo2L/TRAIL is a soluble fragment of the extracellular domain of Apo2 ligand, corresponding to amino acid residues 114-281, available from Genentech, Inc./Amgen/Immunex), an Apo2L/TRAIL DR5 agonist (e.g. apomab that is a fully human monoclonal antibody that is a DR5-targeted pro-apoptotic receptor agonist, as described, for example, in US 2007/0031414 and US 2006/0088523, available from Genentech, Inc.), systemic hedgehog antagonist, erlotinib (TARCEVA™), cetuximab, ABX-EGF, canertinib, EKB-569 and PKI-166), or dual-EGFR/HER-2 inhibitor such as lapatanib. Additional second medicaments include alemtuzumab (CAMPATH™), FavID (IDKLH), CD20 antibodies with altered glycosylation, such as GA-101/GLYCART™, oblimersen (GENASENSE™), thalidomide and analogs thereof, such as lenalidomide (REVLIMID™), ofatumumab (HUMAX-CD20™), anti-CD40 antibody, e.g., SGN-40, and anti-CD80 antibody, e.g. galiximab.

Additional molecules that can be used in combination with the IGF-1R antibodies herein for treatment of cancer include pan-HER tyrosine kinase inhibitors (TKI) that irreversibly inhibit all HER receptors. Examples include such molecules as CI-1033 (also known as PD183805; Pfizer), GW572016 and GW2016 (GlaxoSmithKline) and BMS-599626 (Bristol-Meyers-Squibb).

Additionally included is an inhibitor of apoptosis protein (IAP) antagonist such as, for example, Jafrac2, Diablo/Smac, and other inhibitors described, for example, in Vucic et al., Biochem. J. 385:11-20 (2005).

Also included as second medicaments for cancer treatment are c-Met inhibitors such as, for example, a monoclonal antibody to c-Met such as METMAB™ (a recombinant, humanized, monovalent monoclonal antibody directed against c-Met produced by Genentech, Inc., the variable region sequence of which is described in US 2006/0134104), as well as one-armed formats of METMAB™ antibody such as that described in US 2005/0227324, anti-HGF monoclonal antibodies, truncated variants of c-Met that act as decoys for HGF, and protein kinase inhibitors that block c-Met induced pathways (e.g., ARQ197, XL880, SGX523, MP470, PHA665752, and PF2341066).

Additional such second medicaments for cancer treatment include poly(ADP-ribose) polymerase 1 (PARP) inhibitors such as, for example, KU-59436 (KuDOS Pharma), 3-aminobenzamide (Trevigen, Inc.), INO-1001 (Inotek Pharmaceuticals and Genentech), AG014699 (Pfizer, Inc.), BS-201 and BS-401 (BiPar Sciences), ABT-888 (Abbott), AZD2281 (AstraZeneca), as described, for example, in Nature, 434: 913-917 (2005) and Nature, 434: 917-921 (2005) on the role for PARP inhibition in the development of targeted cancer therapy.

Also included are MAP-erk kinase (MEK) inhibitors such as, for example, U0124 and U0126 (Promega), ARRY-886 (AZD6244) (Array Biopharma), PD 0325901, CI-1040 (Pfizer), PD98059 (Cell Signaling Technology), and SL 327.

Further included are phosphatidylinositol 3-kinase (P13K) inhibitors such as described, for example, in WO 2007/030360, such as LY294002 and wortmannin. Further examples include analogs of 17-hydroxywortmannin (see, e.g., US 2006/0128793), azolidinone-vinyl benzene derivatives, which are described, for example, in WO 2004/007491, and 2-imino-azolinone-vinyl fused-benzene derivatives, which are described, for example, in WO 2005/011686.

Also included are, for example, AKT (protein kinase B) inhibitors such as, for example, SR13668 (SRI International), AG 1296, A-443654, KP372-1, perifosine (also known as KRX-0401; Keryx Biopharmaceuticals), and others such as those described in WO 2006/113837 (for example, imidazo[4,5-c]pyridine analogs with Akt (PKB) kinase antagonist activity containing a 4-amino-1,2,5-oxadiazole substituent at the 2-position of the ring system with an alkyne substituent at the 4-position, and diverse functionality at the 6-position), 1L-6-hydroxymethyl-chiro-inositol 2(R)-2-O-methyl-3-O-octadecylcarbonate, PI (phosphatidylinositol) analogs, a peptide derived from the proto-oncogene TCL1, which binds to the same region on the PH domain as PIP₃, compounds that inhibit by preventing the activation of Akt via inhibition of upstream effectors such as Akt Inhibitor IV, Akt Inhibitor V, and TRICIRIBINE™ (6-amino-4-methyl-8-(β-D-ribofuranosyl).

An alternative approach to blocking PI3K/Akt signaling is the use of small molecules that inactivate the kinase mammalian target of rapamycin (mTOR), which functions downstream of Akt. Three mTOR inhibitors being tested in clinical trials for patients with breast cancer and other solid tumors are CCI-779 (otherwise known as temsirolimus; Wyeth, Madison, N.J.), RAD001 (also known as everolimus; Novartis, New York, N.Y.), and AP23573 (Ariad, Cambridge, Mass.)

Further included are inhibitors of heat-shock protein 90 (HSP90), a chaperone protein that in its activated form controls the folding of many key signal transduction client proteins including HER2, for example, for patients with HER2-overexpressing breast cancer. Examples of HSP90 inhibitors include SNX-5422 (Serenex), geldanamycin and its derivatives such as 17-allylamino-17-demethoxygeldanamycin (17-AAG), pyrazole HSP90 inhibitor CCT0180159 (The Institute of Cancer Research), and tanespimycin (KOS-953) (Kosan Biosciences).

Additional compounds include trastuzumab (HERCEPTIN™) combined with a toxin such as the fungal toxin maytansinoid (DM-1), also called T-DM1 or Herceptin DM1.

Further second medicaments include agents that lower IGF-I concentrations such as growth-hormone releasing hormone (GHRH) antagonists (Letsch et al., Proc Natl Acad Sci USA, 100:1250-1255 (2003)), and a PEGylated GH receptor antagonist (pegvisomant) useful to disrupt GH signaling in patients with acromegaly and cancer (McCutcheon et al., J. Neurosurg., 94: 487-492 (2001)). IGF-I neutralizing monoclonal antibodies and IGFBPs are also useful second medicaments in breast cancer (Van den Berg et al., Eur J Cancer, 33: 1108-1113 (1997)) and prostrate cancer (Goya et al., Cancer Res, 64: 6252-6258 (2004)).

In a preferred combination embodiment for cancer, the antibodies herein are given with another biological agent such as an antibody or another non-chemotherapeutic agent such as an anti-estrogen inhibitor or other targeted inhibitor, more preferably a biological agent or anti-estrogen inhibitor. It is expected that an anti-estrogen inhibitor in combination with an antibody herein may show additive or even synergistic effects in treating breast cancer, particular ER-positive breast cancer.

The antibodies herein can be administered concurrently, sequentially, or alternating with the second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as the first medicaments, or from about 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

### Articles of Manufacture

In another embodiment of the invention, articles of manufacture containing materials useful for the treatment of the disorders described above are provided. In one aspect, the article of manufacture comprises (a) a container comprising the antibodies herein (preferably the container comprises the antibody and a pharmaceutically acceptable carrier or diluent within the container); and (b) a package insert with instructions for treating the cancer in a patient where the patient's cancer expresses one or more of the biomarkers as identified herein.

In a preferred embodiment, the article of manufacture herein further comprises a container comprising a second medicament, wherein the antibody is a first medicament. This article further comprises instructions on the package insert for treating the patient with the second medicament, in an effective amount.

The second medicament may be any of those set forth above, with an exemplary second medicament for cancer being another antibody, chemotherapeutic agent (including cocktails of chemotherapeutic agents), cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, and/or growth-inhibitory agent.

In this aspect, the package insert is on or associated with the container. Suitable containers include, e.g., bottles, vials, syringes, etc. The containers may be formed from a variety of materials such as glass or plastic. The container holds or contains a composition that is effective for treating the disorder in question and may have a sterile access port (e.g., the container may be an intravenous solution bag or a vial having a stopper pierceable by a hypodermic injection needle). At least one active agent in the composition is the antibody herein. The label or package insert indicates that the composition is used for treating the particular disorder in a patient or subject eligible for treatment with specific guidance regarding administration of the compositions to the patients, including dosing amounts and intervals of antibody and any other medicament being provided. Package insert refers to instructions customarily included in commercial packages of therapeutic products that contain information about the indications, usage, dosage, administration, contra-indications, and/or warnings concerning the use of such therapeutic products.

The article of manufacture may further comprise an additional container comprising a pharmaceutically acceptable diluent buffer, such as bacteriostatic water for injection (BWFI), phosphate-buffered saline (PBS), Ringer's solution, and/or dextrose solution. The article of manufacture may further include other materials desirable from a commercial and user standpoint, including other buffers, diluents, filters, needles, and syringes.

In another aspect, the invention provides a method for packaging or manufacturing an antibody herein or a pharmaceutical composition thereof comprising combining in a package the antibody or pharmaceutical composition and a label stating that the antibody or pharmaceutical composition is indicated for treating patients with a cancer.

### Methods of Advertising

The invention herein also encompasses a method for advertising an antibody herein or a pharmaceutically acceptable composition thereof comprising promoting, to a target audience, the use of the antibody or pharmaceutical composition thereof for treating a patient or patient population with cancer characterized by expression of one or more biomarkers as herein disclosed, particularly where the cancer is breast cancer or colorectal cancer.

Advertising is generally paid communication through a non-personal medium in which the sponsor is identified and the message is controlled. One specific form of advertising is through providing a package insert with the pharmaceutical product herein which instructs the user thereof to treat patients who have been identified as candidates for therapy based on expression of biomarkers as disclosed herein, where the patient has cancer, and, in particular, breast cancer or colorectal cancer.

Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the treatment methods herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed, generally in public places. More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments. The advertising may be individualized or customized based on user characterizations defined by service interaction and/or other data such as user demographics and geographical location.

The following are non-limiting examples of the methods and compositions of the invention. It is understood that various other embodiments may be practiced, given the general description provided above. The disclosures of all citations in the specification are expressly incorporated herein by reference.

### Example

To identify biomarkers, a large panel of breast and colorectal cancer cell lines with detailed accompanying molecular genetic characterization were evaluated. A key finding of this study is that IGF-1R receptor levels have predictive value in vitro, specifically that low expression is usually associated with lack of response, and that high levels of the adaptor proteins IRS1 and IRS2 may be positive predictive factors. These in vitro findings were confirmed in vivo studies showing that an IGF-1R antagonist has antitumor activity in xenografted tumor models with either high levels of IGF-1R or the ligand IGF-II, suggesting that pathway focused panels of biomarkers have clinical utility. In addition, unbiased analysis of gene expression data revealed a transcriptional signature predictive of response from the colorectal cancer cell lines. A relationship between IGF-1R expression and ER status in breast cancer was functionally validated and supports rationally designed combination therapy.

### Materials and Methods

### IGF-1R Screening

Cell lines described in this study were obtained from commercial sources and have been described previously (O'Brien et al., Cancer Research 68(13):5380-5389 (July 2008); Wagner et al., Nature Medicine 13(9):1070-1077 (September 2007)), with the exception of BT474EEI, a derivative of BT474 derived by subculturing BT-474 tumors grown in vivo in the absence of estrogen pellet supplementation (EEI, exogenous estrogen independent) as described previously (Lewis Phillips et al., Cancer Research 68(22):9280-9290 (November 2008); US2009/0098,115 A). All breast cancer cell lines were plated out at 3000 cells per well, colorectal lines were plated out between 1000 and 3000 cells per well (depending on growth properties) in 10% fetal bovine serum (FBS) normal media and allowed to settle and recover overnight. The following day the cells were washed in 0% FBS phenol red free media. The cells were then serum starved for 5 hours in 0% FBS phenol red free media. After serum starvation 0%, 0.1%+50 ng/mL IGF-1 or 2.5% FBS was added back to the plates and the cells were dosed with IGF-1R antibody (10H5) starting at a final concentration of 10 ug/mL with 1:3 serial dilutions across the plate. Data for the 2.5% screening condition is shown in FIGS. 34 and 35. Cells were incubated at 37° C. for 72 hours then assayed by CTG.

### Immunoblotting

### Western Blotting Experiments we Conducted Using Standard Protocols.

The blotting antibodies used were IRS1 (Cell Signaling Technology, CST #2382), pIRS1(CST #2384), pAKT(CST #9271), AKT(CST #9272), MAPK(CST #9102), pMAPK(CST #9101), CyclinD1(SC-20044), pS6(CST #2211), p27(BD Bioscience, BD-610241), p4EBP1(CST #9451), pIGF-1R(CST #3024) and IGF-1R(CST #3027). Quantitation of immunoblot bands was accomplished using NIH Image J software. Signal intensity was normalized between lanes by normalization to total Akt and total Erk/1/2. The IP westerns were done against IGF-1R (Genentech #10F5) using the Protein G Immunoprecipitation Kit (Sigma #IP-50). 50 μg of protein was loaded into the column then the Sigma protocol was followed. Mouse IgG (Sigma #15381) was used as a control in all experiments. The blottting antibodies used were pIGF-1R(CST #3021), pIGF-1R(CST #3024) and IGF-1R(CST #3027).

IGF-1R and ESR1 siRNA

All siRNA was done in phenol red free media with 10% FBS. OnTARGET Plus™ small interfering RNA (siRNA) specific to human IGF-1R (Dharmacon, Lafayette, Colo., USA Cat. #L-003012-00), ESR1 (Dharmacon, Lafayette, Colo., USA Cat. #L-003012-00) or a control siRNA that does not target any sequence in the human genome (non-target control, NTC, Dharmacon Cat. #D-001810-10) were used in transient transfection experiments. For IGF-1R and ESR1 knockdown the following siRNAs were used Human IGF-1R; ON-TARGET-PLUS™ Set of 4 LQ-003012-00-0010, Human IGF-1R; ON-TARGET-PLUS SMART-POOL™ L-003401-00-0010, Human ESR1; ON-TARGET-PLUS™ Set of 4LQ-003401-00-0010, Human ESR1.Optimal siRNA duplex and lipid concentrations were determined for each cell-line. For the adherent cell line MCF7 cells were plated at 8000 cells per well in a 96 well plate with 0.25 uL of LIPOFECTIMINE™ RNAiMAX (Cat. #13778-150 Invitrogen, Carlsbad, Calif.) and 25 nM of siRNA per well. Cells were incubated for 3 days in siRNA then 10H5 (IGF-1R antibody) was added for 3 days, followed by addition of Cell Titer Glo. A duplicate plate was made for each cell line, no drug was added and RNA was collected using Qiagen TURBO-CAPTURE™ 96 mRNA Kit (Cat# 72251). mRNA was directly converted to cDNA using ABI cDNA archive kit (ABI, Cat# 4322171). For qRT-PCR analysis cDNA was diluted 1:10 and was mixed with TaqMan Universal PCR Master Mix (ABI, Cat# 4304437) and one of the following 20X primer probes: PPIA Hs99999904_ml (housekeeping gene), UBC Hs00824723_ml (housekeeping gene), ESR1 Hs01046818_ml, IGF-1R Hs00609566_ml, Analysis was done using the delta delta CT method normalizing to the housekeeping genes and then NTC control siRNA treated cells.

### Gene Expression Profiling Studies

Breast and colorectal cancer cell lines were profiled on Affymetrix HGU133P 2.0 as previously described and microarray containing the data used to generate the colorectal h10H5 sensitivity signature has been deposited in the Gene Expression Omnibus (GEO) database under accession numbers GSE12777 (breast data) and GSE8332 (Colorectal data). Microarray data was analyzed using Spotfire and Cluster/Treeview software. Gene differentially expressed between sensitive and resistant colorectal cell lines were identified using the Cyber T algorithm, a modified t-test that uses a Bayesian estimate of variance (Baldi and Long, Bioinformatics 17(6):509-519 (June 2001)), and false discovery rates (FDR) were estimated by the q-value method of Storey and Tibshirani (Storey and Tibshirani, PNAS 100(16):9440-9445 (August 2003)). Cell lines were binned into sensitive and resistant classes using a cutoff of 20% growth inhibition (i.e. cells that showed greater than 20% inhibition in response to 1 μg/mL h10H5 were classified as sensitive). Comparison to published cancer gene expression signatures was performed in the Oncomine database (Rhodes et al., Neoplasia 9(2):166-180 (February 2007)).

### Tumor Xenograft Studies

Female nu/nu or irradiated Balb/c nude mice were inoculated subcutaneously with Colo205 or MCF7 tumor cells, respectively, or female nu/nu mice were inoculated with CXF-280 explant tumor fragments. Once tumors reached a mean volume of 130-260 mm³, mice were then randomized into groups of 8 to 10 mice and treated with vehicle or h10H5 at 1, 5, 15 or 20 mg/kg through intraperitoneal injections. Tamoxifen was given as 5 or 10 mg 60-day slow release drug pellets that were embedded subcutaneously. Tumor volumes were measured in two dimensions (length and width) using UltraCal-IV calipers (Fred V. Fowler Company, Newton, Mass.). The following formula was used with Excel v11.2 to calculate tumor volume: Tumor Volume (mm³)=(length·width²)·0.5.

### Immunohistochemistry (IHC)

The formalin fixed and paraffin-embedded specimens were sectioned at 5 micron onto slides. After deparaffinization and rehydration, sections were processed for IGF-IR IHC analysis. Antigen retrieval was performed using preheated Trilogy buffer (Cell Marque, Rocklin, CA) at 99° C. for 30 minutes. Endogenous peroxidase activity was quenched with KPL Blocking Solution (KPL, Gaithersburg, Md.) at room temperature for 4 minutes. Endogenous avidin/biotin was blocked with Vector Avidin Biotin Blocking Kit (Vector Laboratories, Burlingame, Calif.). Subsequently, sections were incubated with 2.5 μg/ml mouse anti-IGF-IR (clone 5E3, Genentech, CA) monoclonal antibody in blocking serum for 60 minutes at room temperature, and followed by incubation with biotinylated secondary horse anti-mouse antibody for 30 min. Streptavidin conjugated horseradish peroxidase was applied for 30 min and signals were further enhanced by tyramide amplification. Metal Enhanced DAB (Pierce Biotechnology. Rockford, Ill.) was used to develop the slides.

### Results

Activity of an Anti-IGF-1R antibody in breast cancer models and association of IGF-1R expression with Estrogen Receptor status

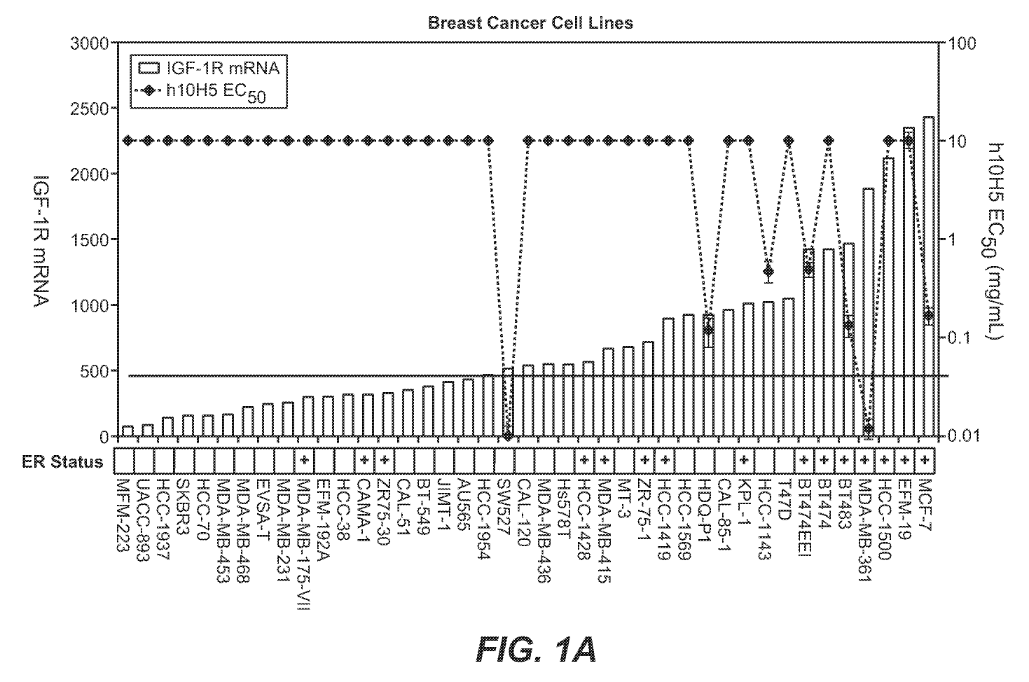

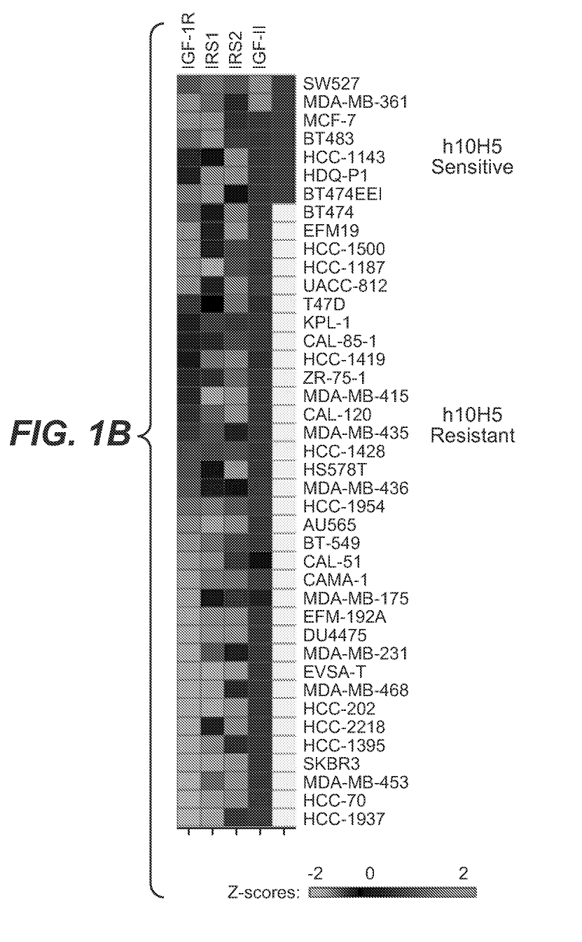

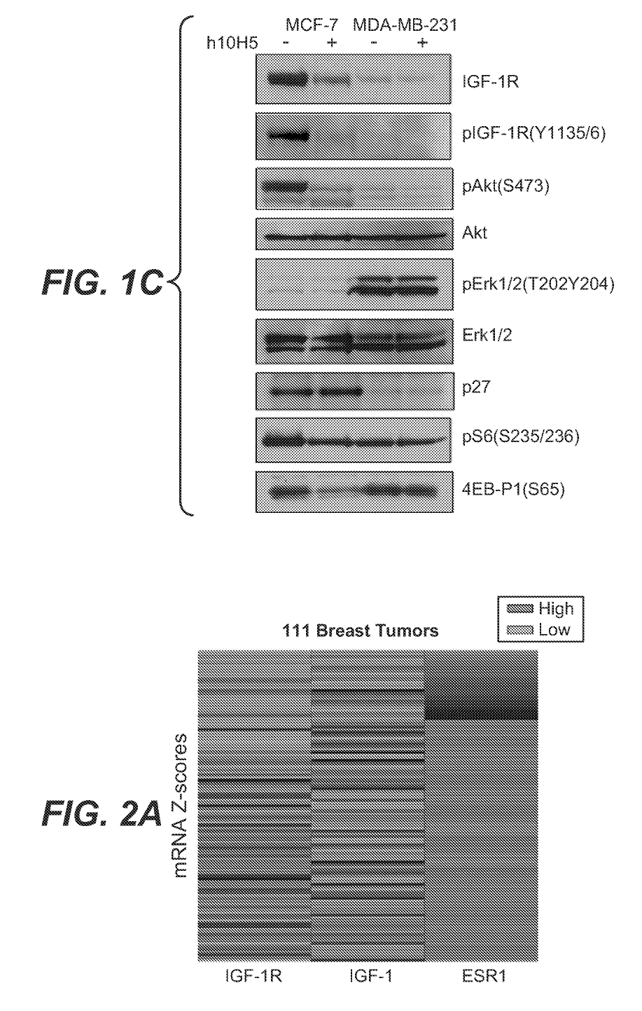

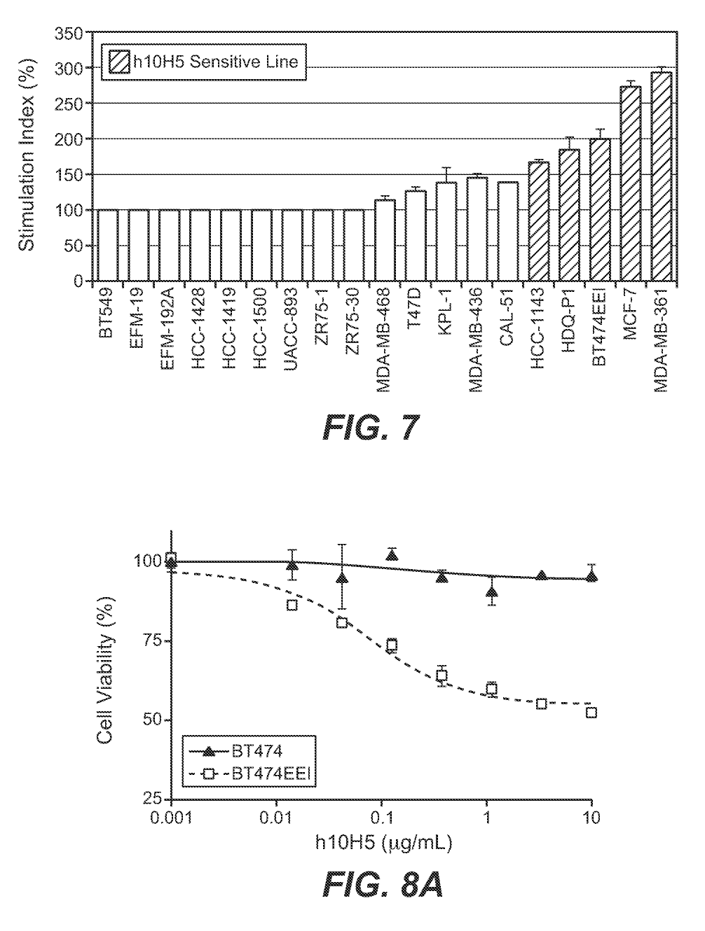

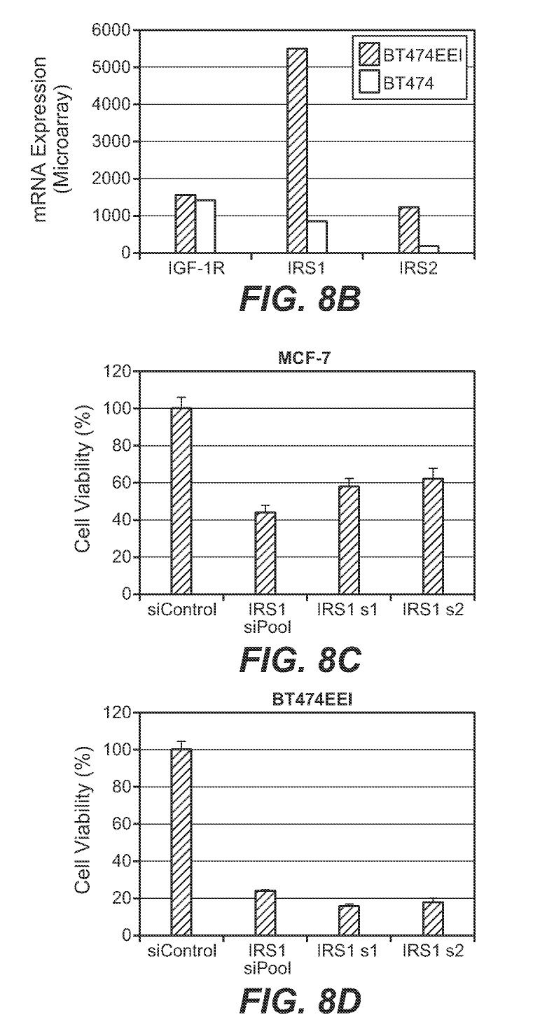

Forty one breast cancer cell lines were assayed for in vitro sensitivity to the humanized recombinant anti-IGF-1R antibody h10H5, as measured in a three day ATP-based cell viability assay. Seven of the 41 cell lines were found to be sensitive, with EC50 values below 1 μg/mL (FIG. 1A). Lack of sensitivity was associated with low expression of the IGF-1R itself, since only 1 out of 21 cell lines with expression below the median level for the panel was sensitive, whereas 6 out of 20 cell lines with IGF-1R expression above the median were sensitive to h10H5 (p=0.05, Fisher's exact test). Thus while the positive predictive value of IGF-1R above median is relatively low at 28%, the negative predictive value of IGF-1R expression below median is 95%. This is consistent with a hypothesis wherein a minimal level of expression of IGF-1R is required for sensitivity to a biotherapeutic targeting this receptor, but where expression alone is not sufficient to confer sensitivity. To investigate the role of the IGF signaling axis further in these cell lines, an IGF-I stimulation index was also determined, defined as the percent increase in cell growth of cells cultured in 1 ng/ml IGF-1 compared to cells grown in serum free media, for a subset of the breast cancer cell lines. IGF-1 was most potent at stimulating cell growth in cells that show in vitro response to h10H5, whereas most non-responsive cell lines had little or no proliferative response to IGF-1 stimulation (FIG. 7). This suggests a model wherein only a subset of breast cancer cells have a functional IGF-I/IGF-1R signaling axis that is linked to the cell cycle machinery and can respond to ligand driven cellular proliferation, and where cellular response to anti-IGF-1R targeting therapies is only effective in the context of an active signaling pathway. Additional molecular predictors of response to h10H5 and pathway activation in breast cancer were identified using gene expression microarray data, since receptor expression alone could account for only approximately one third of the sensitivity, but were unable to identify any additional genes whose expression was associated with sensitivity in a statistically significant manner based on a false discovery rate below 10%. The relationship between expression of IRS1 and IRS2 and h10H5 response across the cell line panel was evaluated in a directed manner (FIG. 1B). Through this analysis it was determined that with the exception of SW527 (which expresses high levels of the ligand IGF-II, FIG. 1B), all of the sensitive cell lines expressed moderate to high levels of IGF-1R as well as high levels of either IRS1 or IRS2 (FIG. 1B). IRS1 and IRS2 are thought to have partially overlapping cellular functions since overexpression of IRS2 in IRS1 null mouse embryonic fibroblasts can reconstitute IGF-1 activation of PI 3-kinase and immediate-early gene expression to the same degree as expression of IRS1 and also partially restores IGF-1 stimulation of cell cycle progression (Bruning et al., Molecular and Cellular Biology 17(3):1513-1521 (March 1997)). In addition, a derivative of the BT474 cell line derived by in vivo passaging, BT474EEI (Lewis Phillips et al., supra), showed marked sensitivity to h10H5 that is not seen in the parental line (FIG. 8). Supervised analysis of gene expression differences between these two lines identified IRS1 overexpression as one of the most dramatic differences between these cell lines (FIG. 8B), which otherwise are quite similar, again consistent with the hypothesis that high levels of IRS effector function are essential to enable cellular responsiveness to h10H5. This model predicts that high levels of IRS1 and IRS2 are important in determine whether the IGF/IGF-1R signaling pathway is coupled to extracellular signaling and thus whether the pathway is active in a given cell line, in which case the function of these genes should be required for cellular proliferation in response to IGF-1. To test this siRNA mediated knockdown of IRS1 was used and showed significant decreases in cell viability under IGF-1 driven growth conditions in the high IRS1 expressing cell lines, MCF-7 and BT474EEI (FIGS. 8C and 8D), suggesting that this adaptor plays an important role in proliferation in response to extracellular signals. Together these results suggest that a multiplex panel of biomarker assays focused on detecting levels of IRS1, IRS2 and IGF-1R might have utility in predicting response to anti-IGF-1R targeting therapies in breast cancer.

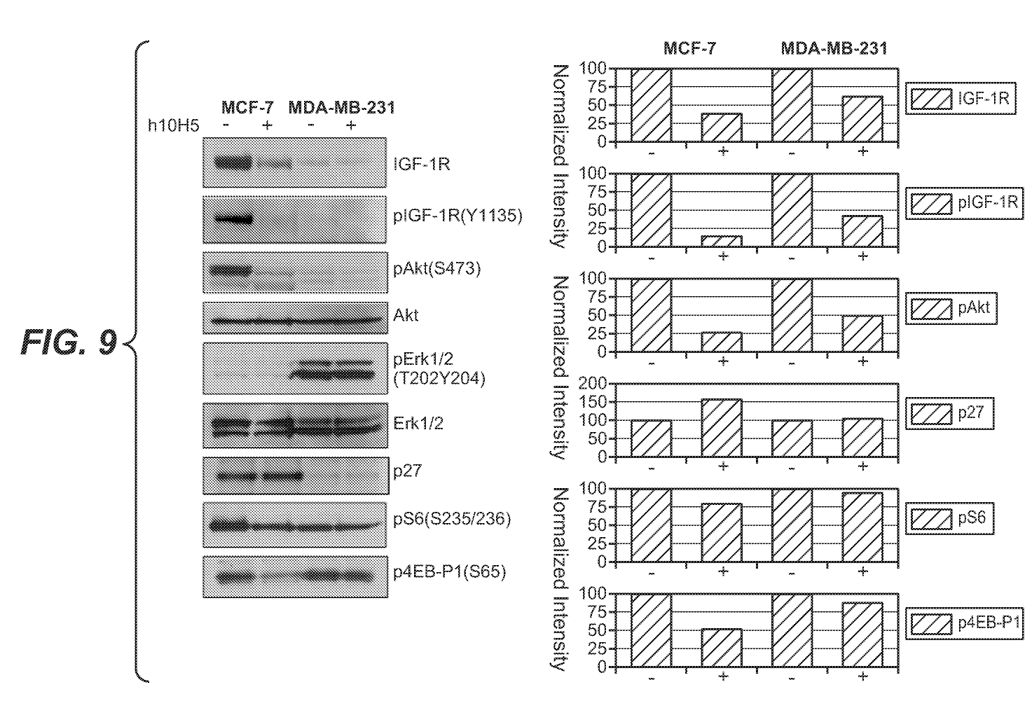

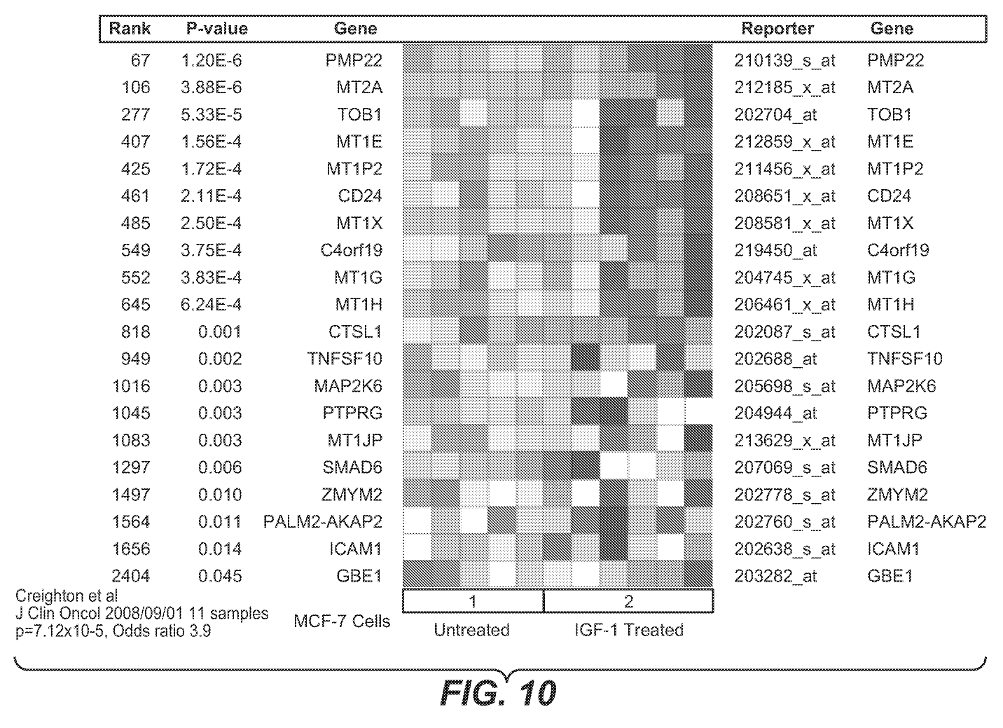

The analyses described above have all focused on the identification of predictive biomarkers that could be used to select patients for therapy based on analyses of archival tumor tissue or pre-treatment biopsies, but we were also interested to identify putative pharmacodynamic biomarkers that might potentially allow assessment of drug activity by comparison of pre- and post-treatment biopsies. To address this both sensitive MCF-7 cells and resistant MDA-MB-231 cells were tested with h10H5 for 24 hours and then examined levels of key signaling proteins and their phosphorylated isoforms by Western blotting. In resistant MDA-MB-231 cells we detected low levels of IGF-1R and observed h10H5 treatment caused downregulation of total and phosphorylated receptor, as well as decreases in pAkt(S473), but minimal effects on distal markers such as pS6 or p4EB-P1. Similar analyses in sensitive MCF-7 cells treated with h10H5 also showed downregulation of total and phosphorylated receptor, as well as decreases in pAkt(S473), suggesting that these proteins and phosphoproteins might have utility as biomarkers of target modulation. In contrast to the MDA-MB-231 results, h10H5 treatment in MCF7 cells resulted in a 50% increase of the negative cell cycle regulator p27 and a 50% decrease in levels of phospho-4EB-P1 (S65) (FIG. 1C and FIG. 9), suggesting that distal outputs of the PI3K/Akt pathway on cell cycle and translational components may correlate with efficacy in response to h10H5 treatment. Assays for such analytes might thus be used to monitor patient response to anti-IGF-1R therapies, potentially providing an early indication of therapeutic benefit and also giving information on optimal biological doses for such therapies.

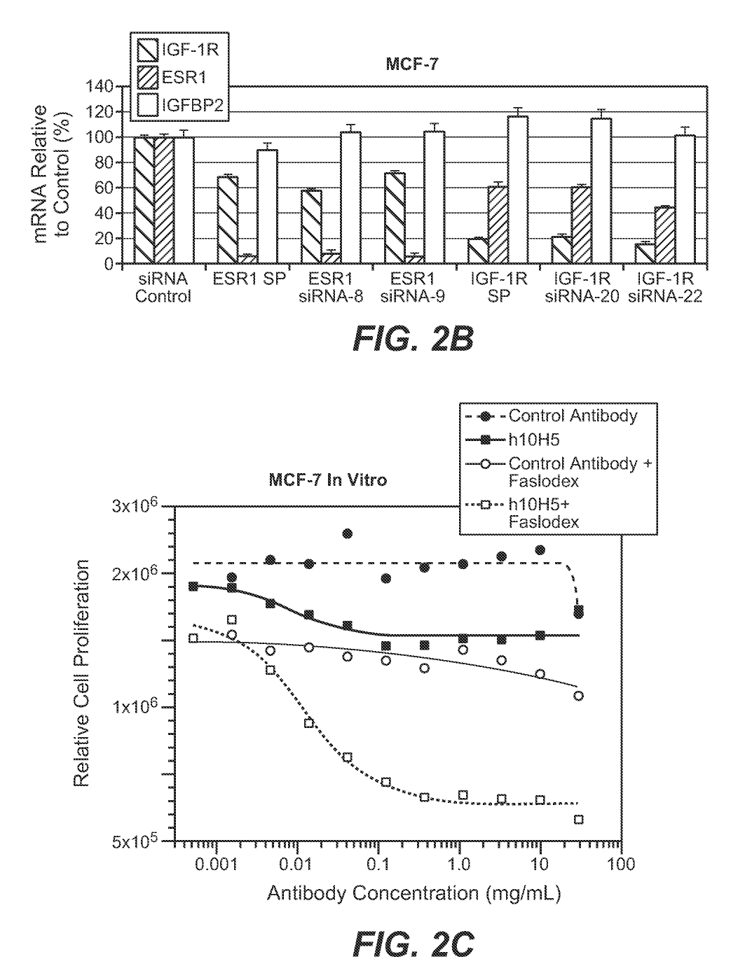

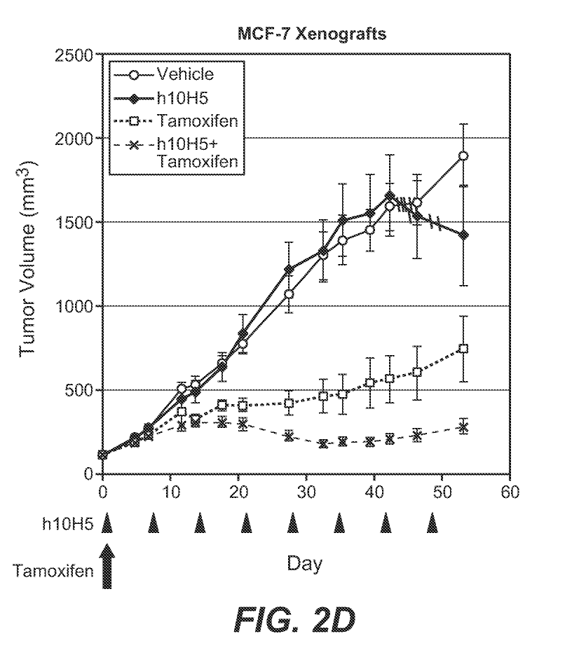

Because breast cancer molecular subtypes are relatively well understood and provide a framework for other targeted therapies (e.g. tamoxifen or aromatase inhibitors in ER positive breast cancer), experiments were designed to determine whether the IGF-1R pathway was associated with particular breast cancer subtypes and whether this might provide a contextual basis for developing anti-IGF-1R therapies in breast cancer. In particular, high IGF-1R expression was associated with estrogen receptor (ER) status, since 13 out of 21 cell lines with IGF-1R expression above the median were ER positive, but only 3 out of 20 cell lines with below median IGF-1R expression were ER positive (P=0.003, Fisher's exact test). To confirm that this association was not a cell line specific phenomenon, microarray data from 111 human breast tumors was analyzed for expression of IGF-1R, IGF-I and estrogen receptor (encoded by the ESR1 gene)—high IGF-1R expression was significantly associated with ESR1 transcript levels in this data set (p<0.001, Wilcoxon rank sum test) (FIG. 2A). IGF-1R is a member of the “intrinsic set” of breast cancer subtype classifier genes and is associated strongly with the luminal, hormone receptor positive subtype (Sorlie et al., PNAS 98(19):10869-10874 (September 2001)). The functional relationship between ER and IGF-1R expression in breast cancer, as well as the consequences of dual blockade of these pathways on cell viability was next evaluated. First, siRNA mediated knockdown of both ESR1 and IGF-1R in estrogen receptor positive MCF-7 cells using both siRNA pools as well as two individual siRNA duplexes was performed. qRT-PCR analysis of lysates prepared from these cells showed that the siRNAs targeting each gene efficiently knocked down their respective targets (FIG. 2B). Each of the ESR1 siRNAs resulted in a 30-40% reduction in IGF-1R levels and each of the IGF-1R siRNAs resulted in 40-50% reduction in ESR1 levels. These results suggest that IGF-1R transcript levels are positively regulated either directly or indirectly by the estrogen receptor, and ESR1 levels are likewise regulated by IGF-1R receptor signaling, and are consistent with previous reports suggesting extensive crosstalk between these pathways (Yee and Lee, Journal of Mammary Gland Biology and Neoplasia 5(1):107-115 (January 2000)). One implication of this finding is that therapeutic agents such as FASLODEX® (fulvestrant) Injection or tamoxifen that target estrogen receptor can enhance the effects of anti-IGF-1R antibodies on cell viability. To test in vitro combination studies with h10H5 and fulvestrant were performed under both normal FBS and media conditions as well as in phenol red free media with charcoal stripped FBS, since previous studies have suggested that phenol red can act as an estrogen mimetic and FBS may contain traces amounts of estrogens (Murphy et al., European Journal of Cancer & Clinical Oncology 25(12):1777-1788 (December 1989)). Consistent with this, growth of MCF-7 cells is substantially more inhibited in the phenol red free charcoal stripped FBS than in normal media, suggestive of the presence of estrogens obscuring response to h10H5 in normal media. In addition, the addition of fulvestrant to h10H5 resulted in substantially greater inhibition of cell growth than either single agent alone (FIG. 2C). The synergistic interaction between h10H5 and anti-estrogen targeting therapeutics in nude mice harboring subcutaneously implanted MCF-7 xenograft tumors was confirmed in vivo (FIG. 2D). In this experiment, once weekly h10H5 had no detectable tumor growth inhibition at the dose and schedule examined, perhaps reflective of the fact that in vivo propagation of these tumors requires estrogen pellets, and consistent with in vitro studies showing that estrogen signaling upregulates IGF-1R and may mask the effects of an IGF-1R targeting antibody. However, significantly greater tumor growth inhibition was observed when tamoxifen was combined with h10H5 (p<0.001) compared to tamoxifen alone, suggesting that dual targeting of these pathways results in greater anti-tumor effects than either single agent alone (FIG. 2D).

Activity of an Anti-IGF-1R Antibody in Colorectal Cancer Models and Association of IGF-1R Expression with Efficacy

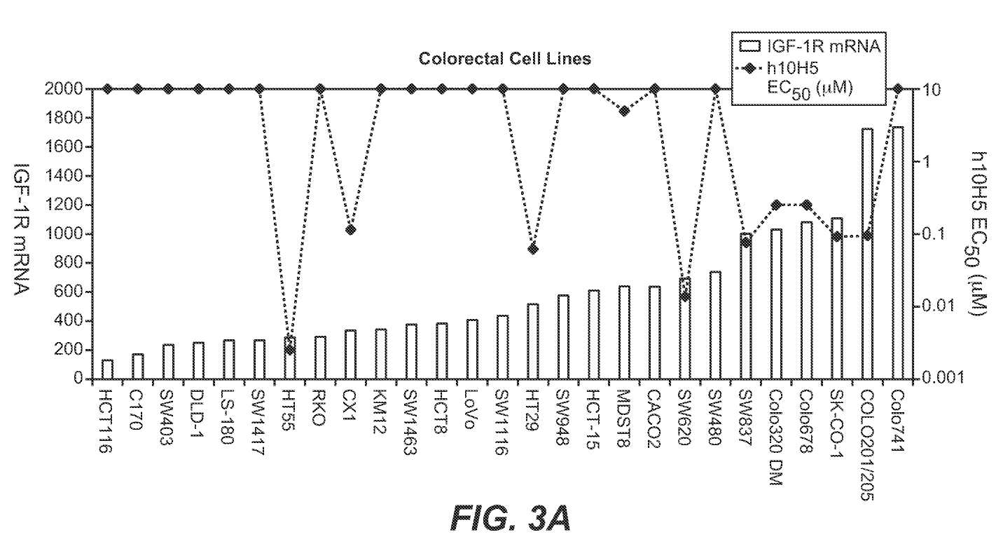

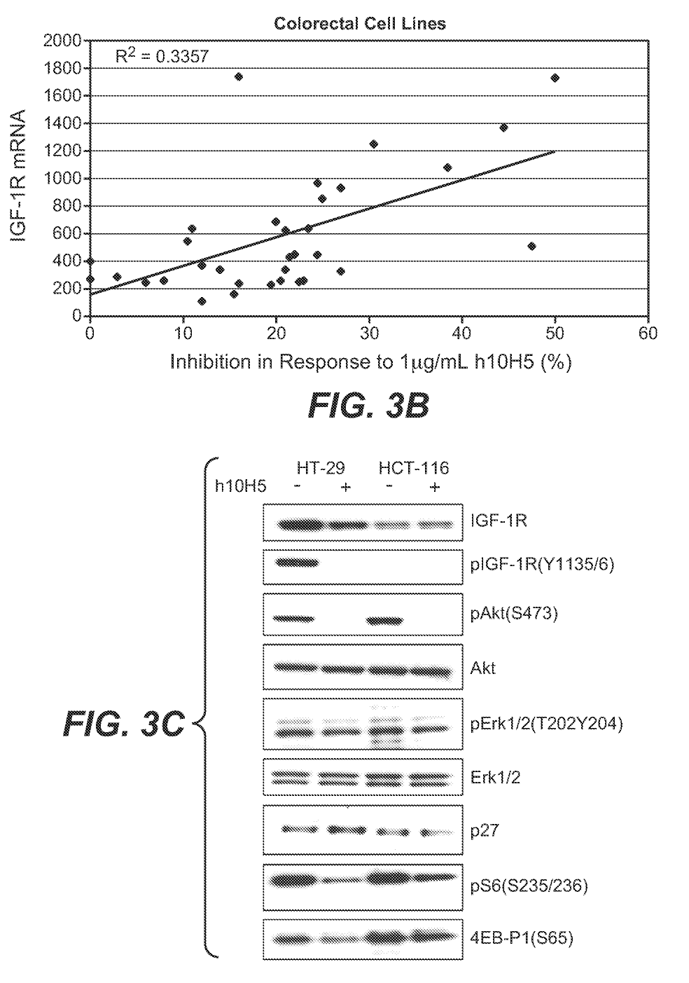

The responsiveness of a panel of 27 colorectal cell lines to h10H5 was evaluated in an effort to identify molecular correlates of response in this tumor type (FIG. 3A). Overall, 9 of the 27 cell lines were sensitive and had EC₅₀ values of less than 1 μg/mL, suggesting relatively strong dependence on IGF-1R signaling in this tumor type. IGF-1R expression itself showed a trend towards higher levels in sensitive models, since seven of 13 cell lines with IGF-1R expression above the median for the panel were sensitive compared to only two cell lines with expression below the median. The negative predictive value was not as strong as seen in breast cancer and the trend did not reach statistical significance. Overall expression levels of IGF-1R were correlated with percent inhibition in response to h10H5 (R²=0.33, FIG. 3B), again suggesting possible diagnostic utility of receptor levels and consistent with previous reports that levels of IGF-1R are correlated with mitogenicity, transformation and adhesion phenotypes (Guvakova and Surmacz, Experimental Cell Research 231(1):149-162 (February 1997)); Rubini et al., Experimental Cell Research 230(2):284-292 (February 1997)). The pharmacodynamic response to h10H5 in sensitive and resistant colorectal models and observed similar results to those in breast cancer was evaluated. Substantial h10H5-mediated downregulation of pAkt(S473) in both sensitive HT-29 cells and resistant HCT-116 cells (FIG. 3C) was observed, but more pronounced effects were seen on distal markers such as p27, pS6 and p4E-BP1 specifically in the sensitive cell line (FIG. 3C).

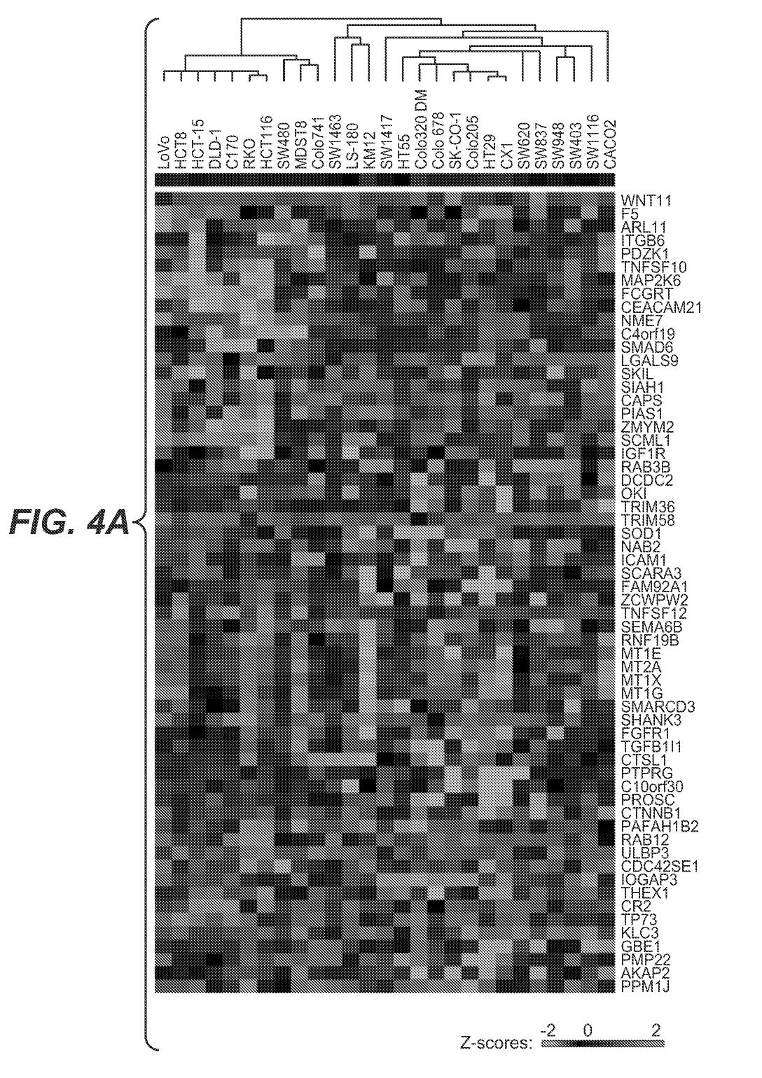

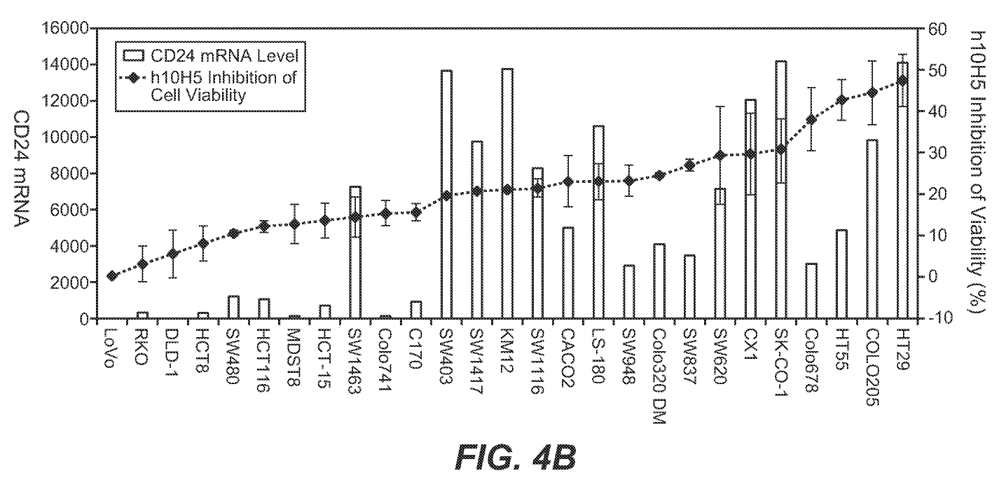

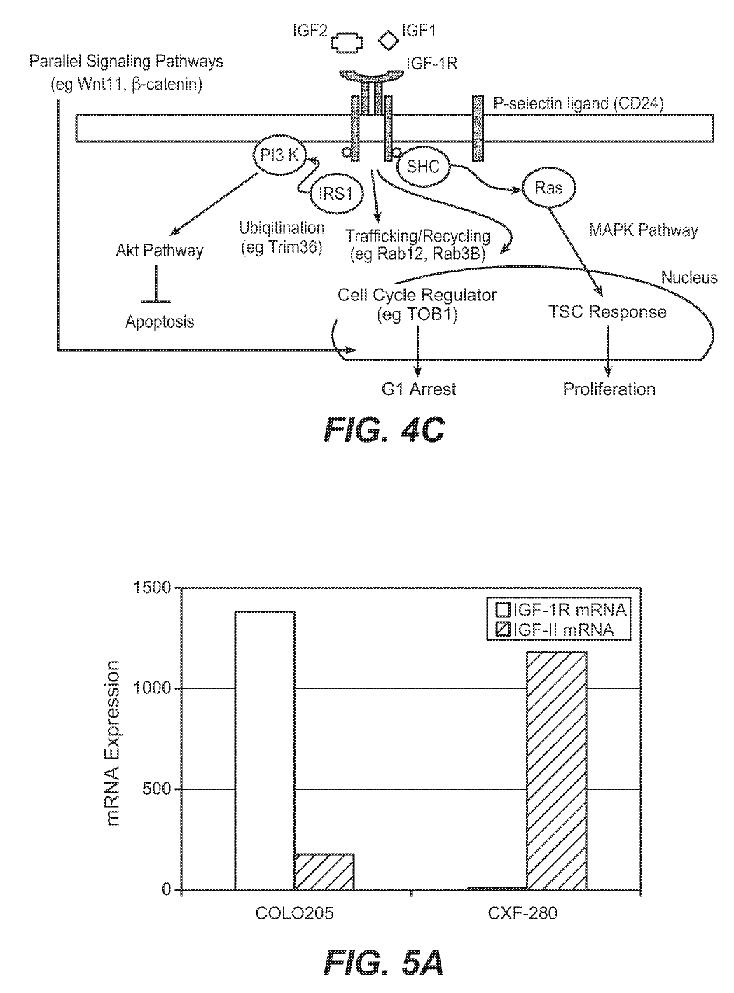

Because IGF-1R levels alone do not explain all of the sensitivity and resistance seen in colorectal cell lines, a molecular signature of anti-IGF-1R response by supervised analysis of gene expression microarray data was identified. Cell lines were binned into sensitive and resistant classes using a cutoff of 20% growth inhibition. This effort led to the identification of 75 probes corresponding to 60 genes that are differentially expressed between sensitive and resistant lines with a false discovery rate of <10% (FIG. 4A). Reassuringly, IGF-1R itself was identified through this unbiased analysis as one of the top genes predicting sensitivity. In addition, pathway analysis implicated components of Wnt signaling such as Wnt-11 and β-catenin as negative predictive factors in response, suggesting that activation of parallel signaling pathways may render cells less sensitive to the inhibitory effects of anti-IGF-1R antibodies. This analysis also identified factors that regulate ubiquitination (e.g. Trim36) and trafficking such as Rab family members, as well as negative regulators of the cell cycle such as Tob1, as additional candidate biomarkers of response. Finally, the P-selectin ligand CD24 also showed significant positive association with h10H5 sensitivity (FIGS. 4A and 4B). Expression of CD24 has been shown to be a poor prognostic marker in colorectal cancer (Weichert et al., Clinical Cancer Research 11(18):6574-6581 (September 2005)) and to be associated with a cancer stem cell phenotype (Vermeulen et al., PNAS 105(36):13427-13432 (September 2008)), suggesting a possible role for IGF-1R targeting in a clinically important subpopulation of colorectal cancer. Based on this it is intriguing to note that a recent report showed that colorectal cancer models selected for resistance to 5-FU or oxaliplatin manifest a stem-cell like phenotype and enhanced sensitivity to an anti-IGF-1R targeting antibody (Dallas et al., Cancer Research 69(5):1951-1957 (March 2009)). To assess the relationship of this colorectal response signature to other published gene expression signatures the ONCOMINE™ database, a compendium of 18,000 cancer related gene expression microarrays (Rhodes et al., Neoplasia 9(2):166-180 (February 2007); Rhodes et al., Neoplasia 9(5):443-454 (May 2007)) was queried. This analysis assesses overlap between the query signature and signatures in the database by generating 2×2 contingency tables and then performing a Fisher's exact test to assess statistical significance between the datasets. This database with the CRC h10H5 response signature revealed a highly significant relationship (p=7.12×10-5) to a published dataset from MCF-7 breast cancer cells treated with IGF-1 (Creighton et al., Journal of Clinical Oncology 26(25):4078-4085 (September 2008)). Components of the signature such as TOB1, CD24, MAP2K6 and SMAD6 were all found to be downregulated upon IGF-I treatment (FIG. 10), suggesting that expression of these putative markers not only correlates with anti-IGF-1R activity but also are functionally impacted by signaling through the pathway, strengthening the rationale for evaluation of this signature a potential predictor of patient response to anti-IGF-1R targeted therapies.

### In Vivo Anti-Tumor Activity of H10H5 in Colorectal Cancer Models

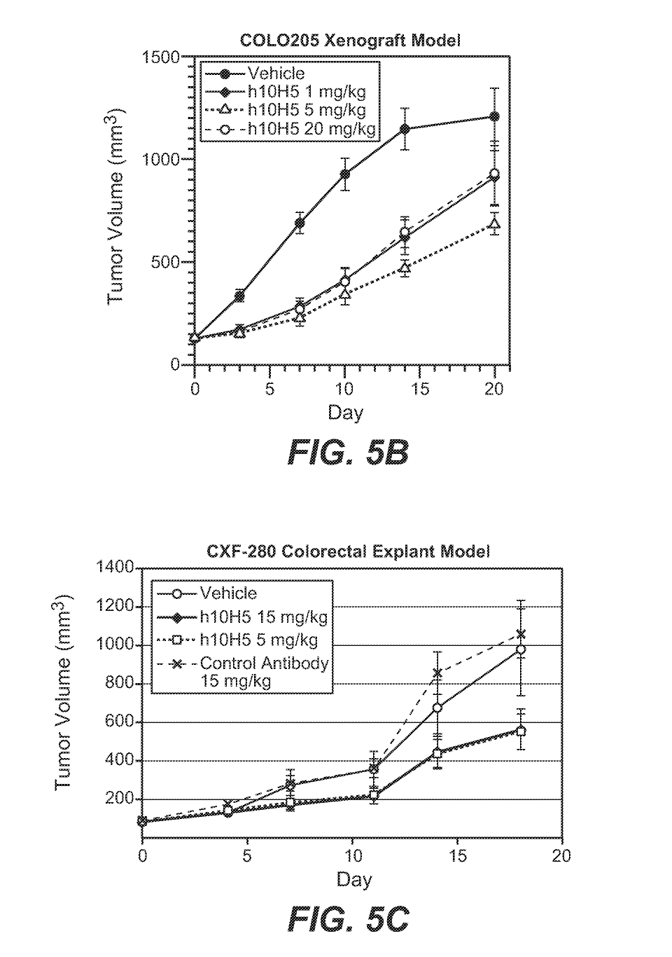

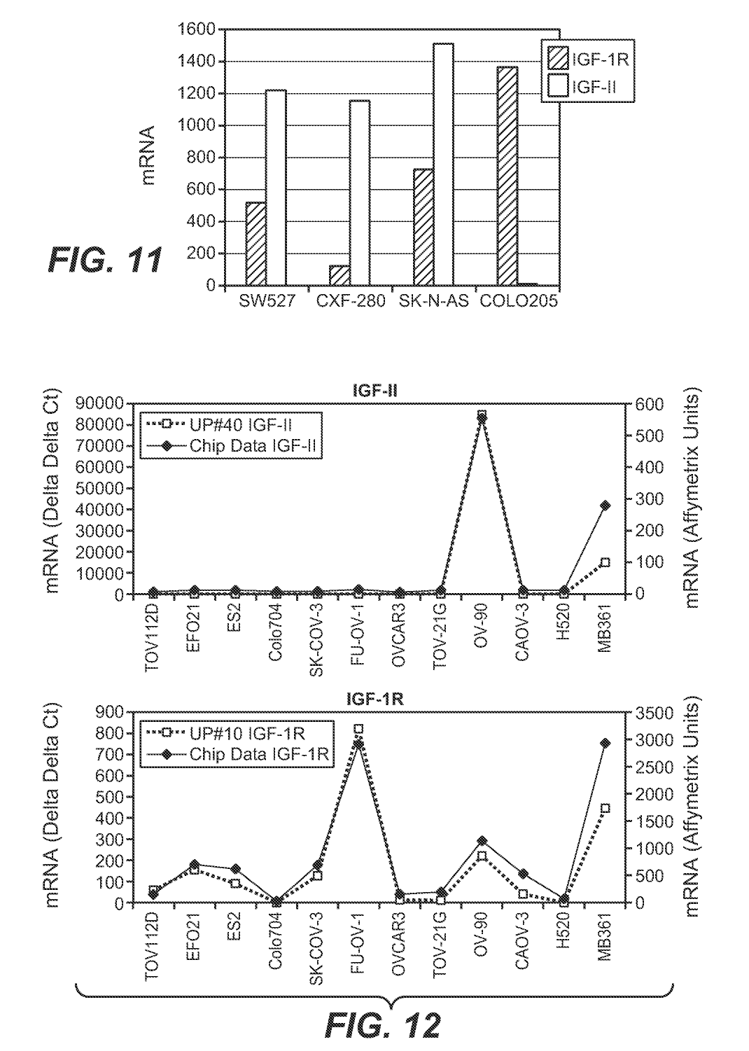

In vivo confirmation of h10H5 activity in both high IGF-1R and high IGF-II expressing models was evaluated by selecting select representative xenograftable cell lines or tumor explants to test each hypothesis. First, h10H5 activity in nude mice harboring subcutaneously implanted Colo-205 xenograft tumors was tested, since this model expresses high levels of IGF-1R (FIG. 5A) and is sensitive to the effects of h10H5 in vitro. Significant tumor growth inhibition at an h10H5 dose of 20 mg/kg was seen in this model (FIG. 5B), providing in vivo proof of concept that anti-IGF-1R antibodies may show benefit in colorectal cancers expressing high receptor levels. Colorectal cancers also frequently express high levels of IGF-II ligand, so h10H5 was evaluated for antitumor activity in primary tumor explant model CXF-280, which expresses high levels of IGF-II but low levels of IGF-1R (FIG. 4A). Such models are derived from patient tumors that have been transplanted subcutaneously directly into nude mice. They are reported to have maintained their typical tumor histology, including a stromal component and vasculature (Fiebig et al., Cancer Genomics Proteomics 4(3):197-209 (May-June 2007)), and hence may be somewhat more representative of actual patient tumors than xenografted cell lines. h10H5 at doses of 5 or 15 mg/kg substantially reduced tumor growth compared to vehicle or a control antibody in CXF-280 explants (FIG. 5B) and also significantly delayed time to tumor progression for both doses of h10H5 compared to control antibody treated animals (Log rank p-value=0.03 for 15 mg/kg group, p=0.02 for 5 mg/kg group). In addition, anti-tumor activity of h10H5 has previously been demonstrated in tumor xenograft models of the breast tumor cell line SW527 and the neuroblastoma cell line SK-N-AS (Shang et al., Molecular Cancer Therapeutics 7(9):2599-2608 (September 2008))—both of these models express high levels of IGF-II (FIG. 11), again suggesting a role for receptor targeting in situations where tumor growth may be driven by autocrine growth loops involving IGF-II. These data indicate anti-IGF-1R directed biotherapeutics have activity in tumors that express components of the signaling pathway and support pathway-focused diagnostic tests for patient selection.

### Development of Pathway Focused Anti-IGF-1R Diagnostic Tests

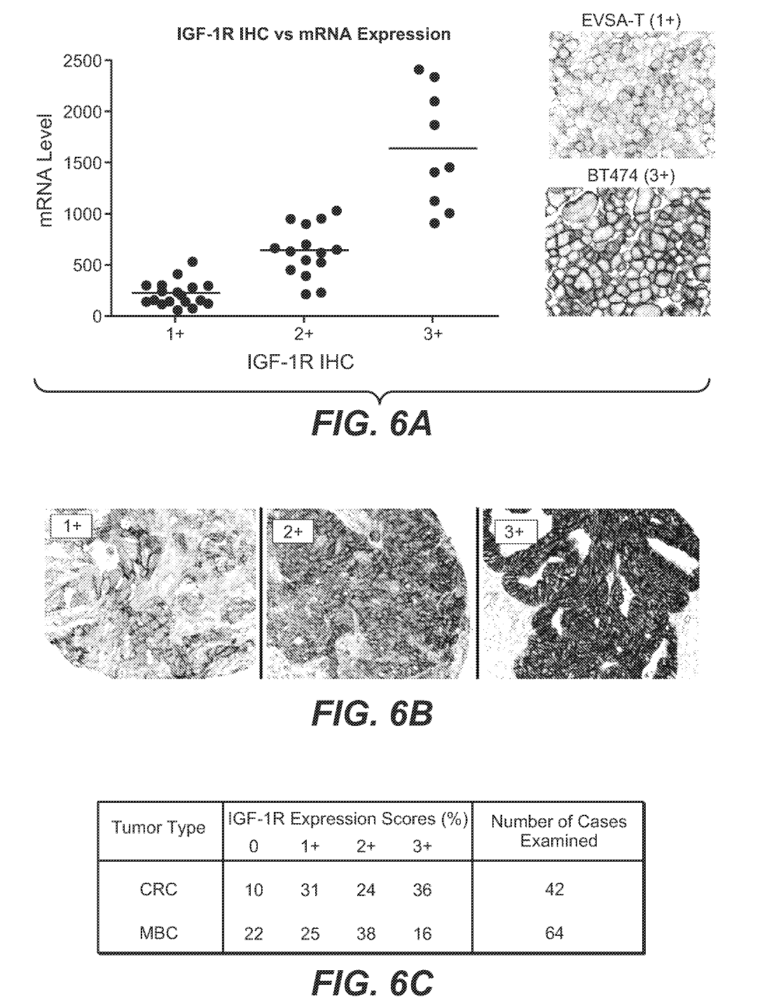

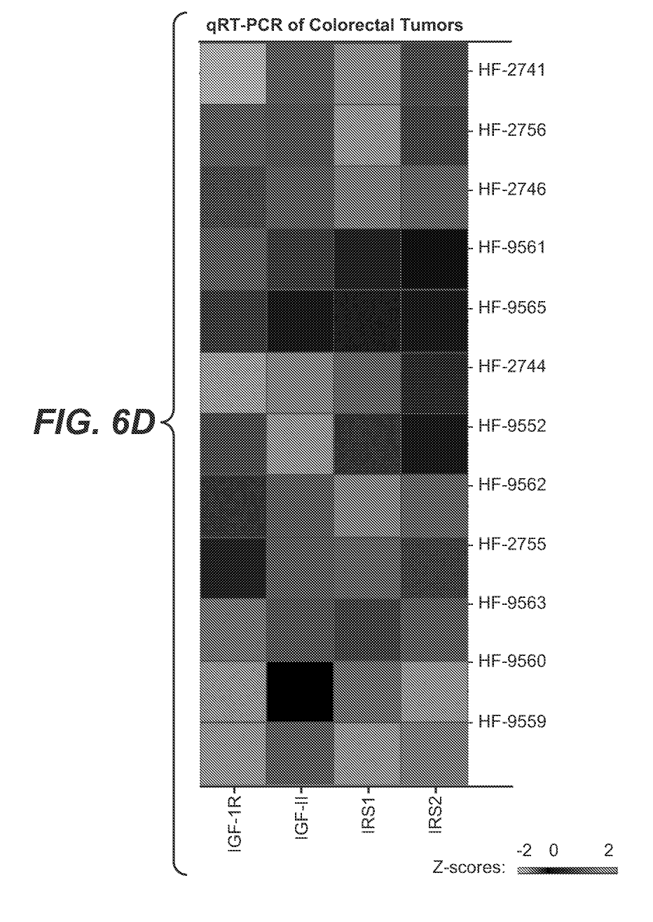

An IHC assay was developed for patient stratification. Initial validation was done on a tissue microarray constructed from formalin fixed paraffin embedded cell pellets derived from 42 breast cancer cell lines for which accompanying gene expression microarray data was available. This allowed comparison of IGF-1R mRNA levels in each cell line with protein staining intensity determined by IHC (FIG. 6A) and showed overall excellent agreement between these two different methods of determining target levels, suggesting the IHC assay is faithfully reading out IGF-1R levels. The assay was next used on a series of breast and colorectal tumor samples and showed that in both tissues a wide range of IGF-1R expression is detectable by this assay, with 60% of colorectal samples and 54% of breast cancer samples exhibiting strong staining (IHC 2+ or 3+). Thus this NC assay may be a valuable tool for evaluating IGF-1R levels as a patient stratification biomarker in clinical samples. Because our studies also implicate components of IGF-1R signaling such as IGF-II and the adaptors IRS1 and IRS2, a multiplex qRT-PCR assay was developed that may be used to assess levels of all of these biomarkers in formalin fixed paraffin embedded tumor specimens. The multiplex assay was validated using control formalin fixed paraffin embedded (FFPE) cell pellet RNA and comparison to microarray data from matched samples (FIG. 12). The assay was applied to RNA prepared from FFPE colorectal tumor material and showed a wide range of expression of these potential biomarkers (FIG. 6D), suggesting that such an assay could be used to clinically test the hypotheses that high expression of IGF-1R and IRS1 or high expression of IGF-II might identify responsive patients.

### DISCUSSION

The major aim of this study was to identify predictive diagnostic biomarkers to help inform patient stratification efforts during clinical development of an anti-IGF-1R antibody in solid tumor malignancies, in particular breast and colorectal cancer. Preclinical studies in well characterized panels of cell lines and tumors were used to evaluate putative predictive biomarkers based on close connection to the pathway biology of IGF-1R signaling, and also to identify novel biomarkers using unbiased pharmacogenomic analysis. These studies have yielded insights into the potential diagnostic utility of the target itself (IGF-1R) as well as key ligands and associated molecules (IGF-II, IRS1, IRS2), and in addition have identified a gene expression signature associated with response in colorectal cancer.

The data above suggest that in breast cancer in particular expression of IGF-1R is necessary but not sufficient for anti-tumor activity, since none of the cell line models with low IGF-1R expression (i.e. equal to or below 1+ on our IHC scale) showed significant inhibition in response to h10H5. Thus stratification of patients based on IGF-1R levels may have utility in identifying patients unlikely to respond due to weak pathway activity.

The results herein suggest that an additional important factor in response to anti-IGF-1R targeting agents is expression of high levels of either of the substrate molecules IRS1 and IRS2. These studies provide confirmation in a broad panel of cell lines that IRS levels in conjunction with IGF-1R may have value as a biomarker of anti-IGF-1R response and support the evaluation of a composite diagnostic test based on tumor expression of key pathway components.

Another diagnostic strategy suggested by our results in breast cancer would be enrichment for patients with high IGF-1R expressing tumors by focusing clinical development on estrogen receptor positive cancers, based on the observation that high IGF-1R expression occurs predominantly in this subset of breast cancer. Thus simply focusing on a disease subtype might be a surrogate approach to screening directly for receptor levels. Such a strategy also has appeal based on the observed in vitro and in vivo synergy between h10H5 and estrogen targeting agents.

The results in cell lines suggest that monitoring pre- and post treatment levels of total and phosphorylated IGF-1R as well as phospho-Akt (S473) in patient biopsies or on CTCs might also have utility in monitoring target pathway modulation in patients treated with anti-IGF-1R targeting biotherapeutics. In addition, these results suggest that monitoring levels of downstream readouts of the IGF-1R axis such as p27 and 4EB-P1 could have value as an early indicator of patient response to therapy, since modulation of these proteins is associated with efficacy in preclinical models.

The foregoing written description is considered to be sufficient to enable one skilled in the art to practice the invention. The present invention is not to be limited in scope by the example presented herein. Indeed, various modifications of the invention in addition to those shown and described herein will become apparent to those skilled in the art from the foregoing description and fall within the scope of the appended claims.

What is claimed is:

1. A method of treating cancer in a human patient comprising administering an IGF-1R inhibitor to the patient, provided the patient's cancer has been shown to express, at a level above the median for the type of cancer being treated, two or more biomarkers selected from the group consisting of IGF-1R, IGF-II, IRS1 and IRS2.

2. The method of claim 1 wherein the patient's cancer expresses IRS1 and/or IRS2 at least one standard deviation above the median.

3. The method of claim 1 wherein the patient's cancer expresses IGF-1R, and either or both of IRS1 or IRS2, above the median.

4. The method of claim 1 wherein the patient's cancer expresses IGF-II, and either or both of IRS1 or IRS2, above the median.

5. The method of claim 1 wherein the cancer is breast cancer.

6. The method of claim 1 wherein the cancer is colorectal cancer.

7. The method of claim 1 wherein the IGF-1R inhibitor is an antibody that binds IGF-1R.

8. The method of claim 7 wherein the IGF-1R antibody is selected from the group consisting of: human antibody, humanized antibody, and chimeric antibody.

9. The method of claim 7 wherein the IGF-1R antibody is selected from the group consisting of: naked antibody, intact antibody, antibody fragment which binds IGF-1R, and antibody which is conjugated with a cytotoxic agent.

10. The method of claim 7 wherein the antibody is selected from the group consisting of: R1507, CP-751,871, MK-0646, IMC-A12, SCH717454, AMG 479, IgG4.P antibody, EM-164/AVE1642, h7C10/F50035, AVE-1642, and 10H5.

11. The method of claim 1 wherein the IGF-1R inhibitor is a small molecule inhibitor.

12. The method of claim 11 wherein the small molecule inhibitor is selected from the group consisting of: INSM-18, XL-228, OSI-906, A928605, GSK-665,602, GSK-621,659, BMS-695,735, BMS-544,417, BMS-536,924, BMS-743,816, NOV-AEW-541, NOV-ADW-742, ATL-1101, and ANT-429.

13. The method of claim 1 wherein biomarker expression has been determined using immunohistochemistry (IHC).

14. The method of claim 1 wherein biomarker expression has been determined using polymerase chain reaction (PCR).

15. The method of claim 14 wherein the PCR is quantitative real time polymerase chain reaction (qRT-PCR).

16. The method of claim 1 wherein a biological sample from the patient has been tested for biomarker expression.

17. The method of claim 16 wherein the biological sample is from a patient biopsy.

18. The method of claim 16 wherein the biological sample is selected from the group consisting of: circulating tumor cells (CTLs), serum, and plasma from the patient.

19. A method of treating breast cancer in a human patient comprising administering an IGF-1R inhibitor to the patient, provided the patient's cancer has not been found to express IGF-1R at a level below the median for breast cancer.

20. A method of treating breast cancer in a human patient comprising administering an IGF-1R inhibitor to the patient, provided the patient has been shown to express one or more biomarkers selected from the group consisting of IGF-1R, IRS1, IRS2, IGF-II, and estrogen receptor, at level above the median for breast cancer.

21. A method of treating breast cancer in a human patient comprising administering a combination of an IGF-1R inhibitor and an estrogen inhibitor, wherein the combination results in a synergistic effect in the patient.

22. The method of claim 21 wherein the IGF-1R inhibitor is an antibody and the estrogen inhibitor is tamoxifen.

23. The method of claim 21 wherein the IGF-1R inhibitor is an antibody and the estrogen inhibitor is fulvestrant.

24. A method for treating a patient with colorectal cancer, comprising administering a therapeutically effective amount of an IGF-1R inhibitor to the patient, provided the patient's cancer expresses IGF-1R at a level greater than the median level for IGF-1R expression in colorectal cancer.

25. A method for treating a patient with colorectal cancer, comprising administering a therapeutically effective amount of an IGF-1R inhibitor to the patient, provided the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1.

26. The method of claim 25 wherein the patient's cancer expresses two ore more of the biomarkers.

27. The method of claim 25 wherein the patient's cancer expresses three or more of the biomarkers.

28. The method of claim 25 wherein the patient's cancer further expresses IGF-1R at a level above the median for colorectal cancer.

29. A method for selecting a therapy for a patient with cancer, comprising administering a therapeutically effective amount of an IGF-1R inhibitor to the patient, if the patient's cancer: has been shown to express, at a level above the median for the type of cancer being treated, two or more biomarkers selected from the group consisting of: IGF-1R, IGF-II, IRS1 and IRS2.

30. A method for selecting a therapy for a patient with breast cancer, comprising administering a therapeutically effective amount of an IGF-1R inhibitor to the patient, provided the patient's cancer:
(a) has not been found to express IGF-1R at a level below the median for breast cancer; or
(b) has shown to express one or more biomarkers selected from the group consisting of IGF-1R, IRS1, IRS2, IGF-II, and estrogen receptor, at level above the median for breast cancer.

31. A method for selecting a therapy for a patient with colorectal cancer, comprising administering a therapeutically effective amount of an IGF-1R inhibitor to the patient, provided the patient's cancer:
(a) expresses IGF-1R at a level greater than the median level for IGF-1R expression in colorectal cancer; or
(b) expresses one or more biomarkers selected from the group consisting of: TOB1, CD24, MAP2K6, SMAD6, TNFSF10, PMP22, CTSL1, ZMYM2, PALM2, ICAM1, and GBE1.

32. An article of manufacture comprising, packaged together, a pharmaceutical composition comprising an IGF-1R inhibitor in a pharmaceutically acceptable carrier and a package insert stating that the inhibitor or pharmaceutical composition is indicated for treating:
(a) a patient with cancer, if the patient's cancer has been shown to express, at a level above the median for the type of cancer being treated, two or more biomarkers selected from the group consisting of: IGF-1R, IGF-II, IRS1 and IRS2;
(b) a patient with breast cancer, if the patient's cancer has not been found to express IGF-1R at a level below the median for breast cancer;
(c) a patient with breast cancer, if the patient's cancer has shown to express one or more biomarkers selected from the group consisting of IGF-1R, IRS1, IRS2, IGF-II, and estrogen receptor, at level above the median for breast cancer;
(d) a patient with colorectal cancer, if patient's cancer expresses IGF-1R at a level greater than the median level for IGF-1R expression in colorectal cancer; or
(e) a patient with colorectal cancer, if the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1.

33. A method for manufacturing an IGF-1R inhibitor or a pharmaceutical composition thereof comprising combining in a package the inhibitor or pharmaceutical composition and a package insert stating that the inhibitor or pharmaceutical composition is indicated for treating:
(a) a patient with cancer, if the patient's cancer has been shown to express, at a level above the median for the type of cancer being treated, two or more biomarkers selected from the group consisting of: IGF-1R, IGF-II, IRS1 and IRS2;
(b) a patient with breast cancer, if the patient's cancer has not been found to express IGF-1R at a level below the median for breast cancer;
(c) a patient with breast cancer, if the patient's cancer has shown to express one or more biomarkers selected from the group consisting of IGF-1R, IRS1, IRS2, IGF-II, and estrogen receptor, at level above the median for breast cancer;
(d) a patient with colorectal cancer, if patient's cancer expresses IGF-1R at a level greater than the median level for IGF-1R expression in colorectal cancer; or
(e) a patient with colorectal cancer, if the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1.

34. A method for advertising an IGF-1R inhibitor or a pharmaceutically acceptable composition thereof comprising promoting, to a target audience, the use of the inhibitor or pharmaceutical composition thereof for treating:
(a) a patient with cancer, if the patient's cancer has been shown to express, at a level above the median for the type of cancer being treated, two or more biomarkers selected from the group consisting of: IGF-1R, IGF-II, IRS1 and IRS2;
(b) a patient with breast cancer, if the patient's cancer has not been found to express IGF-1R at a level below the median for breast cancer;
(c) a patient with breast cancer, if the patient's cancer has shown to express one or more biomarkers selected from the group consisting of IGF-1R, IRS1, IRS2, IGF-II, and estrogen receptor, at level above the median for breast cancer;
(d) a patient with colorectal cancer, if patient's cancer expresses IGF-1R at a level greater than the median level for IGF-1R expression in colorectal cancer; or
(e) a patient with colorectal cancer, if the patient's cancer expresses one or more biomarkers selected from the group consisting of: TOB1, CD24, MAP2K6, SMAD6, TNFSF10, PMP22, CTSL1, ZMYM2, PALM2, ICAM1, and GBE1.
